(19) United States
(12) Patent Application Publication
(10) Pub. No.: US 2023/0149148 A1
(43) Pub. Date: May 18, 2023

(54) SYSTEMS AND METHODS FOR ENTRAPPING AND/ OR REMOVING CLOTS TO PROVIDE BLOOD FLOW RESTORATION IN A VESSEL

(71) Applicant: Squadra Lifesciences, Inc., Palo Alto, CA (US)

(72) Inventors: Salvatore MANGIAFICO, Florence (IT); Mario MUTO, Naples (IT); Mehmet Hakan AKPINAR, Istanbul (TR); Rafal Wojciech LOPALLO, Naples (IT); Antonello FAZIO, Monza (IT); Scott Edward PARAZYNSKI, Houston, TX (US); Thomas NISSL, Mackenrode (DE)

(21) Appl. No.: 18/150,423

(22) Filed: Jan. 5, 2023

Related U.S. Application Data
(63) Continuation of application No. PCT/US2021/042042, filed on Jul. 16, 2021.
(60) Provisional application No. 63/053,045, filed on Jul. 17, 2020.

(51) Int. Cl. A61F 2/01; A61B 17/22

(52) U.S. Cl. CPC A61F 2/013; A61F 2/0105; A61F 2/011; A61B 17/22; A61B 2017/22079; A61B 2017/00022

(57) ABSTRACT

A system used to entrap clots in arteries, such as cerebral arteries, between the artery wall and an external surface of a stent to provide restoration of blood flow therethrough that includes a stent forming an annular wall that defines a set of openings, and an expansion member extendable through the stent to place a distal portion thereof distal to the stent. The stent is advanced into a clot disposed within a vessel and transitioned to an expanded configuration such that the annular wall engages the clot. The expansion member extends through the stent and is transitioned to an expanded state such that a diameter of the distal portion is greater than a diameter of a proximal portion thereof. The expanded distal portion is sized to fill a portion of the vessel to prevent clot fragments from flowing distal to the expansion member.

### CROSS-REFERENCE TO RELATED APPLICATIONS

This application is a continuation of International Patent Application No. PCT/US2021/042042, filed Jul. 16, 2021, and entitled, “Systems and Methods for Entrapping and/or Removing Clots to Provide Blood Flow Restoration in a Vessel,” which claims priority to and the benefit of U.S. Provisional Patent Application No. 63/053,045, filed Jul. 17, 2020, and entitled, “Temporary Endovascular Clot Bypass System for Ischemic Stroke,” the disclosure of each of which is incorporated herein by reference in its entirety.

### BACKGROUND

The present disclosure is related to devices for providing blood flow restoration in a vessel (e.g., in cerebral arteries after ischemic stroke or the like) and a method for manufacturing and using such a device.

Stroke is the leading cause of permanent disability and one of the most frequent causes of dementia in the developed world. Approximately 80% of strokes are ischemic and due to thromboembolic events.

Historically, the first treatment method for acute ischemic stroke has been done with intravenous (IV) tissue-type plasminogen activator (tPA). However, this method has disadvantages like a narrow time window after stroke onset and only a moderate recanalization rate, especially in the proximal arteries. As a result, it has been needed improvement to obtain more successful clinical outcomes in stroke patients. The low success of IV tPA treatment to provide recanalization of large vessel occlusions (LVO) prompted the development of endovascular thrombectomy.

Endovascular thrombectomy techniques can be examined under three main headings: mechanical thrombectomy, aspiration techniques, and tandem techniques.

Mechanical thrombectomy is one of the common treatment methods for strokes. For example, stentriever thrombectomy devices can have high recanalization rates with a reduction in the recanalization time and low complication rates. There are several techniques for implementation of mechanical thrombectomy such as basic stentriever technique, balloon guide catheter technique, tri-axial system/distal access catheter method, distal access catheter method, plain suction thrombectomy, Solumbra technique, intracranial balloon angioplasty, and suction thrombectomy, intracranial stenting for re-occlusion. Moreover, some mechanical thrombectomy devices may be classified into different subtypes based on their mechanism of action such as, for example, coil retrievers, stentrievers, mechanical clot disruption systems (e.g., using laser or ultrasound), and/or the like.

Stentriever thrombectomy devices are self-expandable stent-like devices that are fully retrievable and can have advantages such as providing flow restoration and mechanical thrombectomy. Moreover, the use of stentriever thrombectomy devices is associated with low rates of symptomatic intracerebral hemorrhage and low mortality rates. In addition, the thrombectomy procedure using the stentriever thrombectomy technique can be repeated until the arterial flow is restored with a thrombolysis in cerebral infarction (TICI) grade of 2b or 3.

Aspiration techniques can be used in some specific conditions including, for example, when occlusions are located in the terminal internal carotid artery (ICA), for middle cerebral artery bifurcation and trifurcation thrombi, as well as for hard thrombi that can be resistant to stentriever thrombectomy device recanalization attempts. Aspiration catheters offer an alternative strategy for achieving thrombus removal. The aspiration catheters achieve vascular reperfusion by applying suction at the proximal portion of the occlusion and drawing it into the catheter lumen. The main advantages of the aspiration technique(s) are the fast procedure time and the high rate of favorable clinical outcomes.

Tandem techniques can be used for the recanalization of tandem occlusions. Tandem occlusions are a combination of the extracranial segment of the internal carotid artery (ICA) occlusion with a concurrent occlusion of the intracranial segment. Tandem occlusions are not common but represent challenging therapeutic conditions in the setting of acute ischemic stroke. The intervention in patients with tandem occlusions consists of 2 steps: the first step is revascularization of the extracranial ICA segment by balloon angioplasty or by stent implantation, as in the treatment of atherosclerotic stenosis. The second step is mechanical recanalization of the occluded intracranial artery with the stentriever thrombectomy or aspiration technique. In some instances, faster cerebral flow can result if the distal recanalization can be performed before the recanalization of the ICA.

Challenges persist, however, with current endovascular thrombectomy techniques. For example, the use of some current stentriever thrombectomy devices can result in vascular damage and/or clot fragmentation, which may result in distal embolization and occlusion of previously uninvolved territory. Another challenge with some current stentriever thrombectomy devices is that procedures can result in acute in-stent thrombosis in cases where the stent is permanently left in place following successful recanalization.

Accordingly, a need exists for improved devices, systems, and/or methods for entrapping and/or removing a clot to provide flow restoration in cerebral arteries.

### SUMMARY

In some embodiments, a system for providing blood flow restoration through a target vessel includes a stent and an expansion member. The stent can form an annular wall defining a plurality of openings. The stent is configured to be advanced into and/or at least partially through a clot within a vessel of a human body and transitioned to an expanded state such that the annular wall engages at least a portion of the clot. The expansion member is configured to extend through an interior of the stent such that a distal portion of the expansion member is distal to a distal end of the stent. The expansion member is configured to transition to an expanded state after extending through the stent such that a diameter of the distal portion of the expanded expansion member is greater than a diameter of a proximal portion of the expanded expansion member. The distal portion of expansion member in the expanded state is sized to fill a portion of a lumen of the vessel to prevent clot fragments from flowing through the vessel distal to the distal portion of the expansion member.

### BRIEF DESCRIPTION OF THE DRAWINGS

FIGS. 1 and 2 each is a schematic illustration of a reperfusion system according to an embodiment, and shown in a first configuration or state and a second configuration or state, respectively.

FIG. 3 is a schematic illustration of a reperfusion system according to an embodiment.

FIG. 4 is a schematic illustrations of the reperfusion system of FIG. 3, and showing a free distal prolongation of a stent-graft and a curtain, which can act as a filter during residual thrombus aspiration.

FIG. 5 is a schematic illustration of a portion of the reperfusion system of FIG. 3, and showing a double diameter balloon for stent expansion, a guidewire lumen for advancing the balloon over the guidewire, and a radiopaque marker to show the location of the stent on the balloon surface.

FIG. 6 is a schematic illustration of a portion of the reperfusion system of FIG. 3, and showing a double diameter self-expandable stent structure and a detachable pusher cable to advance and retrieve the stent.

FIG. 7 is a schematic illustration of a portion of the reperfusion system of FIG. 3, and showing a thin membrane to block clot particles to come to the vessel lumen and prevent distal embolization.

FIG. 8 is a schematic illustration of a portion of the reperfusion system of FIG. 3, and showing a double-lumen catheter used to advance the self-expandable stent and/or the balloon over a guidewire via one lumen and to provide aspiration via the other lumen.

FIG. 9 is a schematic illustration of the reperfusion system of FIG. 3 in use after inflating a distal end of the balloon to block the vessel for potential distal embolization.

FIG. 10 is a schematic illustration of the reperfusion system of FIG. 3 showing a clot being moved backward by an internal hydraulic pressure created by the balloon inflation.

FIGS. 11 and 12 are schematic illustrations of the reperfusion system of FIG. 3 showing a process of moving a part of the vessel trapped between the vessel wall and the stent membrane on the outer surface by inflation of the balloon, which can be repeated as desired to move the clot back to the aspiration lumen of the microcatheter (not shown).

FIG. 13 is a schematic illustration of the reperfusion system of FIG. 3, and showing details that the stent is expanded (e.g., into an expanded configuration) and the clot trapped between the stent and the artery wall such that all particles created by a fracture of the main clot are aspirated by the aspiration lumen of the microcatheter leaving no clot either on the distal or the proximal side of the artery that may embolize distally to result in new strokes.

FIG. 14 is a schematic illustration of the reperfusion system of FIG. 3, and showing details that the balloon is deflated to result in recanalization and to obtain blood perfusion.

FIG. 15 is a schematic illustration of the reperfusion system of FIG. 3, and showing details that the balloon catheter is retrieved and the guidewire is maintained in case of another intervention attempt.

FIGS. 16 and 17 are a perspective view and a side view, respectively, of at least a portion of a reperfusion system according to an embodiment.

FIGS. 18 and 19 are a perspective view and a side view, respectively, of a stent and a pusher cable of included in the portion of the reperfusion system of FIG. 16.

FIG. 20 is a flat pattern illustration of the stent of FIGS. 16-19 showing the stent after a manufacturing step of laser-cutting prior to a manufacturing step of heat-setting into a desired shape.

FIGS. 21 and 22 are each a perspective side view illustration of an expansion member according to different embodiments.

FIGS. 23A and 23B are schematic illustrations of a device for sensing a flow of blood through a vessel according to an embodiment, and showing, for example, a temperature distribution associated with no blood flow through the vessel and a temperature distribution associated with blood flowing through the vessel, respectively.

FIG. 24 is a schematic illustration of a device for sensing a flow of blood through a vessel according to an embodiment.

FIG. 25 is a schematic illustration of at least a portion of a reperfusion system according to an embodiment, and shown in use within a vessel.

FIG. 26 is a schematic illustration of a stent configured for use in a reperfusion system, according to an embodiment.

FIG. 27 is a flowchart illustrating a method of using a reperfusion system according to an embodiment.

FIG. 28 is a flowchart illustrating a method of using a reperfusion system according to an embodiment.

### DETAILED DESCRIPTION

One or more embodiments described herein can provide a system, device, and/or method for at least partial blood flow restoration through a vessel via the entrapment and/or removal of a clot. In some implementations, such a system and/or device can be a reperfusion system and/or device for ischemic stroke patients providing flow restoration by recanalization in cerebral arteries. In some implementations, reperfusion of a vessel can include but is not limited to at least one or any suitable combination of bypassing a clot, entrapping a clot, dislodging a clot, aspirating a clot, and/or the like.

In some embodiments, a system for providing blood flow restoration through a target vessel includes a stent and an expansion member. The stent can form an annular wall defining a set of openings. The stent is configured to be advanced into and/or at least partially through a clot within a vessel of a human body and transitioned to an expanded state such that the annular wall engages at least a portion of the clot. The expansion member is configured to extend through an interior of the stent such that a distal portion of the expansion member is distal to a distal end of the stent. The expansion member is configured to transition to an expanded state after extending through the stent such that a diameter of the distal portion of the expanded expansion member is greater than a diameter of a proximal portion of the expanded expansion member. The distal portion of expansion member in the expanded state is sized to fill a portion of a lumen of the vessel to prevent clot fragments from flowing through the vessel distal to the distal portion of the expansion member.

In some embodiments, a system includes a multi-lumen catheter having at least a first lumen and a second lumen, a stent forming a set of cells, and an expansion member. The stent is transitionable between a collapsed state in which the stent is allowed to be advanced from the first lumen of the multi-lumen catheter and at least partially through a clot, and an expanded state in which an outer surface of the stent engages at least a portion of the clot. The expansion member is configured to extend from the first lumen of the multi-lumen catheter through an interior of the stent such that a distal portion of the expansion member is distal to a distal end of the stent. The expansion member is configured to transition to an expanded state to engage a wall of the vessel distal to the clot, thereby preventing clot fragments from flowing through the vessel distal to the distal portion of the expansion member. The second lumen of the multi-lumen catheter is configured to allow continuous aspiration of a lumen of vessel proximal to the distal portion of the expansion member when each of the stent and the expansion member is in the expanded state.

In some embodiments, a method for restoring blood flow through a vessel in a body includes advancing a multi-lumen catheter through the vessel and along a guidewire to a position proximate to a clot in the vessel. A stent is advanced along the guidewire from the multi-lumen catheter and at least partially through the clot. An expansion member is advanced along the guidewire from the multi-lumen catheter and through the clot to place a distal portion of the expansion member distal to the clot. After advancing the stent and the expansion member, the stent is transitioned to an expanded state such that an outer surface of the stent exerts a radially outwardly directed pressure on the clot and the expansion member is transitioned to an expanded state such that the distal portion of the expansion member engages a wall of the vessel distal to the clot. A lumen of the vessel proximal to the distal portion of the expansion member is aspirated via the multi-lumen catheter while each of the stent and the expansion member is in the expanded state.

In some embodiments, a reperfusion system and/or device can include a stent formed of memory shaped materials such as nickel-titanium alloy (e.g., Nitinol®), for example, a nitinol wire, sheet, or tube (e.g., formed into a laser cut, closed cell, open cell, and/or mesh structure). The stent can be a self-expanding, fully deploying, fully retrievable stent. The stent can be sized, shaped, and/or configured for expansion into a clot and/or to facilitate clot retention. The stent can have any suitable deployed diameter (e.g., about 2 mm-10 mm, or larger) and/or deployed length (e.g., about 10 mm-40 mm, or larger).

In some embodiments, a reperfusion system and/or device can include a balloon that may be temporarily inflated to prevent emboli traveling (e.g., in a distal direction) during a procedure.

In some embodiments, a reperfusion system and/or device can include a curtain, filter, and/or thin membrane that can at least partially surround the thrombus and prevent its fragmentation and embolization.

In some embodiments, a reperfusion system and/or device can include an aspiration system that aspirates and extracts a thrombus.

In some embodiments, a reperfusion system can be fully visible under fluoroscopy.

In some embodiments, a reperfusion system and/or device can be configured to lower radial forces associated with the deployment of the stent and/or can include one of more atraumatic features to decrease the risk of vessel wall injury during the procedure.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

In general, terms used herein, and especially in the appended claims (e.g., bodies of the appended claims) are generally intended as “open” terms. For example, the term “including” should be interpreted as “including but not limited to,” the term “having” should be interpreted as “having at least,” etc. Similarly, the terms “comprises” and/or “comprising,” when used in this specification, specify the presence of stated features, integers (or fractions thereof), steps, operations, elements, and/or components, but do not preclude the presence or addition of one or more other features, integers (or fractions thereof), steps, operations, elements, components, and/or groups thereof.

As used herein the term “and/or” includes any and all combinations of one or more of the associated listed items. It should be understood that any suitable disjunctive word and/or phrase presenting two or more alternative terms, whether in the description, claims, or drawings, contemplate the possibilities of including one of the terms, either of the terms, or both terms. For example, the phrase “A or B” will be understood to include the possibilities of “A” or “B” or “A and B.”

All ranges disclosed herein also encompass any and all possible subranges and combinations of subranges thereof unless expressly stated otherwise. Any listed range should be recognized as sufficiently describing and enabling the same range being broken down into at least equal subparts unless expressly stated otherwise. As will be understood by one skilled in the art, a range includes each individual member.

As used herein, the terms “about,” “approximately,” and/or “substantially” when used in connection with stated value(s); geometric feature(s), structure(s), or relationship(s); and/or the like is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 100 can include 90 to 110. Similarly, a first surface may be described as being substantially parallel to a second surface when the surfaces are nominally parallel. While a value, feature, structure, relationship, etc. stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

Similarly, the term “relatively” when used to modify a characteristic is intended to convey that the characteristic so defined is to be considered within and/or relative to the context in which it is discussed and is not necessarily intended to define the characteristic in an absolute and/or global manner, unless expressly stated otherwise. For example, a device that is inserted into the vasculature of a body may be said have a “relatively small size” and thus, is intended to be considered within the context of such devices rather than, for example, the context of very small objects (e.g., microscopic objects, atomic particles, etc.) or very large objects (e.g., planetary objects).

As used herein, the term “set” can refer to multiple features or a singular feature with multiple parts. For example, when referring to a set of walls, the set of walls can be considered as one wall with multiple portions, or the set of walls can be considered as multiple, distinct walls. Thus, a monolithically constructed item can include a set of walls. Such a set of walls may include multiple portions that are either continuous or discontinuous from each other. A set of walls can also be fabricated from multiple items that are produced separately and are later joined together (e.g., via a weld, an adhesive, or any suitable method).

As used herein, the term “stiffness” is related to an object's resistance to deflection, deformation, and/or displacement that is produced by an applied force, and is generally understood to be the opposite of the object's “flexibility.” For example, a wall with greater stiffness is more resistant to deflection, deformation and/or displacement when exposed to a force than a wall having a lower stiffness. Similarly stated, an object having a higher stiffness can be characterized as being more rigid than an object having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of a portion of the object different from the portion of the object to which the force is directly applied. Said another way, in some objects, the point of deflection is distinct from the point where force is applied.

Stiffness (and therefore, flexibility) is an extensive property of the object being described, and thus is dependent upon the material from which the object is formed as well as certain physical characteristics of the object (e.g., cross-sectional shape, length, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity. In another example, the stiffness of the object can be increased or decreased by changing the flexural modulus of a material of which the object is constructed.

Flexural modulus is used to describe the ratio of the applied stress on an object in flexure to the corresponding strain in the outermost portions of the object. The flexural modulus, rather than the modulus of elasticity, is used to characterize certain materials, for example plastics, that do not have material properties that are substantially linear over a range of conditions. An object with a first flexural modulus is less elastic and has a greater strain on the outermost portions of the object than an object with a second flexural modulus lower than the first flexural modulus. Thus, the stiffness of an object can be increased by including in the object a material having a high flexural modulus.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be decreased by decreasing and/or changing the shape of the object.

As used herein, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

In some instances, the words “proximal” or “distal” can be relative terms and do not necessarily refer to universally fixed positions or directions. For example, a distal end portion of a peripheral intravenous (PIV) catheter can be inserted into a vein of a patient's forearm while a proximal end portion of the PIV catheter can be substantially outside of the body. Veins, however, carry a flow of oxygen-poor blood from distal portions of the body back to the heart and, as a result, PIV catheters are generally inserted into a vein such that a distal tip of the PIV catheter is disposed within the vein in a position proximal to the insertion point (e.g., extending relative to the vein in a proximal direction). Thus, a distal position relative to the PIV catheter can refer to, for example, a proximal position relative to the vein (e.g., closer to the heart). Conversely, a distal position relative to a catheter inserted into an artery can refer to, for example, a distal position relative to the artery since arteries carry a flow of oxygen-rich blood from the heart to distal portions of the body.

As used herein, the term “catheter” generally refers to an element configured to define a passageway for accessing a portion of the body (e.g., of a human and/or animal). In some instances, the passageway defined by a catheter can be used for moving fluids (e.g., bodily fluids) or physical objects (e.g., a stent, a guide wire, an expansion member, a punctate plug, a hyaluronic-acid-gel, etc.) from a first location to a second location. In some instances, the passageway defined by a catheter can be used for moving fluids into the body (e.g., fluid delivery) or out of the body (e.g., aspiration or the like).

Any of the devices and/or systems described herein can use one or more catheters to deliver and/or retrieve components of the system and/or to provide aspiration of at least a portion of a target vessel. In some implementations, delivery, retrieval, and aspiration can be provided via a single lumen defined by a catheter. In some implementations, delivery and/or retrieval of the one or more components of the system can be provided via a first lumen of a multi-lumen catheter, and aspiration can be provided via a second lumen of the multi-lumen catheter. In some implementations, delivery and/or retrieval of the one or more components of the system can be provided via a lumen of a first catheter, and aspiration can be provided via a lumen of a second catheter separate from the first catheter. In some such implementations, the first catheter and the second catheter can be disposed, for example, side-by-side (e.g., advanced through and extending from a delivery or introducer catheter). In other such implementations, the first catheter and the second catheter can be, for example, in a coaxial configuration in which one catheter is disposed in and extends through the other. In some implementations, a system can include a multi-lumen catheter having more than two lumens and/or can utilize more than two catheters.

As used herein, the phrase “at least one catheter collectively having at least a first lumen and a second lumen” is intended to refer in general to any of the possible catheter configurations employed by a system. For example, “at least one catheter collectively having at least a first lumen and a second lumen” can refer to a multi-lumen catheter (“at least one catheter”) having at least a first lumen and a second lumen. Alternatively, “at least one catheter collectively having at least a first lumen and a second lumen” can refer to a first catheter and a second catheter (“at least one catheter”) collectively having a first lumen (e.g., defined by the first catheter) and a second lumen (e.g., defined by the second catheter). Moreover, the first catheter and the second catheter can be coaxial or non-coaxial. Accordingly, any of the embodiments and/or systems described herein can include at least one catheter collectively having at least a first lumen and a second lumen and are not intended to be limited in any way to a single or specific implementation unless the context clearly dictates otherwise.

The term “expandable” as used herein may refer to a device or component of a system capable of expanding from a first size or configuration to a second size or configuration. An expandable structure, therefore, is not intended to refer to a structure that might undergo slight expansion, for example, from a rise in temperature or other such incidental cause, unless the context clearly indicates otherwise. Conversely, “non-expandable” should not be interpreted to mean completely rigid or dimensionally stable because some slight expansion, which is typical with known “non-expandable” components, may be observed.

Devices and/or components of the systems disclosed herein are generally capable of transitioning between two or more configurations, states, shapes, and/or arrangements. For example, stents described herein can be “compressible” and/or “expandable” between any suitable number of configurations. Various terms can be used to describe or refer to these configurations and are not intended to be limiting unless the context clearly states otherwise. For example, a stent or an expansion member (or any other component discussed herein) can be described as being placed in a “collapsed state,” which may be any suitable configuration that allows or enables, for example, delivery, retrieval, and/or placement of the stent, expansion member, etc. Examples of collapsed states can include a compressed state, a folded state, a rolled state, a deflated state, a constrained state, and/or similar states or any suitable combinations thereof. Similarly, a stent, expansion member, etc. can be described as being placed in an “expanded state,” which may be any suitable configuration that is not expressly intended for delivery, retrieval, and/or placement of the stent, expansion member, etc. Examples of expanded states can include a released state, a relaxed state, a deployed state, a non-delivery state, and/or similar states or any suitable combinations thereof. While specific examples are provided above, it should be understood that they are not intended to be an exhaustive list of states, configurations, etc. Other states and/or configurations may be possible. Moreover, various terms can be used to describe the same or substantially similar states and/or configurations and thus, the use of particular terms are not intended to be limiting and/or to the exclusion of other terms unless the terms and/or states or configurations are mutually exclusive, or unless clearly stated otherwise.

Any of the embodiments, systems, and/or methods described herein can include a stent that can be formed from a material and/or can otherwise be arranged such that the stent is “self-expanding.” Such a stent, for example, can be formed from a shape-memory alloy or the like and during manufacturing, can be heat-set and/or otherwise biased to or toward an expanded state. As such, an external force exerted on the stent can be operable to constrain and/or otherwise place the stent in a collapsed state, while removal of the external force can be operable to allow the stent to transition to or toward the expanded (i.e., biased) state. Accordingly, the stent can be considered “self-expanding.”

A system can include a self-expanding stent that can be placed in a desired location relative to, for example, a clot in a vessel, and can be allowed to “self-expand” to the expanded state. In some implementations, the system can also include an expansion member such as an inflatable balloon that can be used to facilitate or aid the expansion of the stent or that can be used to “over-expand” the stent. Similarly stated, in some implementations, the expansion member can be used to expand the stent beyond an extent associated with the self-expansion of the stent (e.g., in at least one direction or extent). For example, in some implementations, a stent can be a wire frame or the like that forms a set of cells and that can self-expand into a predetermined configuration. In some implementations, an expansion member or the like can be used to over-expand the stent beyond an extent associated with the self-expansion, which in turn, can increase a radial extent of at least a portion of the stent and can, for example, decrease an axial extent of at least a portion of the stent (e.g., the stent is widened but shortened). In some implementations, over-expanding the stent can include and/or can be the result of portions of the expansion member extending through, for example, the cells of the stent. As described in further detail herein with respect to specific embodiments, the over-expansion of the stent can allow for a desired amount of contact or engagement with a surface of a clot; a desired amount of pressure exerted on at least a portion of the surface of the clot; a staged, gradual, and/or controlled engagement of the clot; and/or the like. Moreover, in contrast to, some known stentriever devices used to capture or retrieve a clot of portions thereof, any of the systems described herein can include a stent that can transition to an expanded state (or an over-expanded state) operable to dislodge a clot, which in turn, can be removed from the vessel via aspiration or any suitable mechanical removal.

The embodiments described herein and/or portions thereof can be formed or constructed of one or more biocompatible materials. In some embodiments, the biocompatible materials can be selected based on one or more properties of the constituent material such as, for example, stiffness, toughness, durometer, bioreactivity, etc. Examples of suitable biocompatible materials include but are not necessarily limited to metals, glasses, ceramics, and/or polymers. Examples of suitable metals include pharmaceutical grade stainless steel (e.g., 316 L stainless steel), gold, titanium, nickel, platinum, tin, chromium, copper, and/or alloys thereof. Moreover, any of the embodiments described herein and/or components thereof can be formed from superelastic or shape-memory alloys such as nickel-titanium alloys (e.g., Nitinol®).

Suitable biocompatible materials may be biodegradable or non-biodegradable. Examples of suitable biodegradable polymers include polylactides, polyglycolides, polylactide-co-glycolides, polyanhydrides, polyorthoesters, polyetheresters, polycaprolactones, polyesteramides, poly(butyric acid), poly(valeric acid), polyurethanes, biodegradable polyamides (nylons), and/or blends and copolymers thereof. Examples of non-biodegradable polymers include non-degradable polyamides (nylons), polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and/or blends and copolymers thereof.

Some biocompatible synthetic material(s) can include, for example, polyesters, polyurethanes, polytetrafluoroethylene (PTFE) (e.g., Teflon™), and/or the like. Where a thin, durable synthetic material is contemplated (e.g., for a covering), synthetic polymer materials such expanded PTFE or polyester may optionally be used. Other suitable materials may optionally include elastomers, thermoplastics, polyurethanes, thermoplastic polycarbonate urethane, polyether urethane, segmented polyether urethane, silicone polyether urethane, polyetheretherketone (PEEK), silicone-polycarbonate urethane, polypropylene, polyethylene, low-density polyethylene (LDPE), high-density polyethylene (HDPE), ultra-high density polyethylene (UHDPE), polyolefins, polyethylene-glycols, polyethersulphones, polysulphones, polyvinylpyrrolidones, polyvinylchlorides, other fluoropolymers, polyesters, polyethylene-terephthalate (PET) (e.g., Dacron®), Poly-L-lactic acids (PLLA), polyglycolic acid (PGA), poly(D, L-lactide/glycolide) copolymer (PDLA), silicone polyesters, polyamides (Nylon), PTFE, elongated PTFE, expanded PTFE, siloxane polymers and/or oligomers, and/or polylactones, and block co-polymers using the same.

Some components and/or portions thereof can be formed of a constituent and/or base material that is coated with one or more polymers. Suitable polymer coatings can include, for example, polyethylene vinyl acetate (PEVA), poly-butyl methacrylate (PBMA), translute Styrene Isoprene Butadiene (SIBS) copolymer, polylactic acid, polyester, polylactide, D-lactic polylactic acid (DLPLA), polylactic-co-glycolic acid (PLGA), and/or the like.

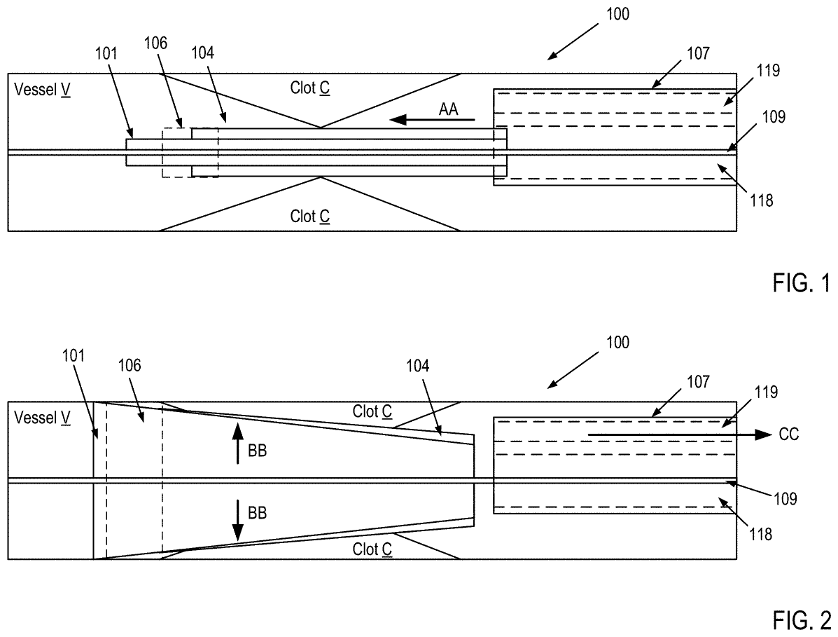

Referring now to the drawings, FIGS. 1 and 2 illustrate a reperfusion system and/or device 100 according to an embodiment, and shown in a first configuration or state and a second configuration or state, respectively. The reperfusion system and/or device 100 (referred to herein as “system 100”) includes a stent 104 and an expansion member 101 that can be delivered to a target location within a vessel V via a catheter 107. For example, as shown in FIGS. 1 and 2, the stent 104 and expansion member 101 are delivered via the catheter 107 to and/or near a location of a clot C within a vessel V. As described in further detail herein, the system 100 is configured to bypass, engage, and/or otherwise remove the clot C to provide restoration of blood flow through the vessel V.

In general, the system 100 can be used to bypass, engage, and/or remove a clot from any vessel within the body. For example, the system 100 can be used to remove a clot from an artery within the human body. In some implementations, the system 100 can be used to remove a clot from a cerebral artery (e.g., after or in response to ischemic stroke). In such implementations, rapid reperfusion of the artery can be important in preventing further tissue damage. The relatively small size of at least some cerebral arteries, however, can present challenges to the safe removal of clots. Moreover, clot fragments flowing distally through the vessel can lead to embolization and/or occlusion of other portions of the vessel. Accordingly, in some embodiments, the system 100 can have a relatively small size operable to allow access to at least some distal cerebral arteries. In addition, the system 100 (e.g., the expansion member 101 and/or other components of the system 100) can be configured to limit and/or substantially prevent the distal flow of clot fragments, thereby reducing risks associated with subsequent embolization and/or occlusion of the vessel.

While the embodiments and methods are described herein as being suitable to treat clots in cerebral arteries, it should be understood that the embodiments and/or methods are not limited to such use unless explicitly stated otherwise. Furthermore, the system 100 can be used to bypass, engage, and/or remove “soft” clots (e.g., relatively new clots with a mucus-like consistency) or “hard clots” (e.g., relatively old clots that have hardened over time).

The stent 104 of the system 100 can be any suitable shape, size, and/or configuration. In some implementations, a size and/or configuration can be based at least in part on a desired use of the system 100. For example, the stent 104 can have a substantially annular shape (e.g., a hollow shell in the shape of a cylinder) corresponding to the substantially tubular shape of the vessel V. In some implementations, the stent 104 can have a relatively small diameter when the system 100 is used to treat and/or remove a clot in relatively small vessels such as, for example, distally located cerebral arteries, when treating a pediatric patient, and/or the like. Alternatively, the stent 104 can have a larger diameter when the system 100 is used to treat and/or remove a clot in larger vessels (e.g., proximally located cerebral arteries and/or other arteries in the body). Moreover, the proximal and distal ends of the stent 104 can be open, which in turn, can allow a portion of the system 100 and/or any other suitable member to be disposed in or at least partially advanced through the stent 104 from a proximal position to a distal position (or vice versa). As shown in FIGS. 1 and 2, such an arrangement can allow the stent 104 to be advanced over and/or along a guidewire 109 (or guidewire catheter) and/or can allow at least a portion of the expansion member 101 to extend through the stent 104, as described in further detail herein.

The stent 104 can be an expandable frame or structure configured to transition between a collapsed configuration and/or state having a first size and an expanded configuration and/or state (FIG. 2) having a second size larger than the first size. As described in further detail herein, the arrangement of the stent 104 in the collapsed or unexpanded state can allow at least a portion of the stent 104 to be inserted in and/or through the clot C (FIG. 1), while the arrangement of the stent 104 in the expanded state can be allow for, result in, and/or otherwise facilitate a desired engagement and/or entrapment of the clot C in the vessel V (FIG. 2). In some embodiments, the stent 104 can be transitioned in response to an applied force such as the inflation of a balloon catheter or the like. Alternatively, the stent 104 can be a self-expanding stent formed from a superelastic and/or shape-memory material such as a nickel-titanium alloy such as Nitinol® and/or the like. In such embodiments, for example, the stent 104 can be laser cut from a sheet or tube of Nitinol® and expanded (e.g., heat-set) into a desired shape, size, and/or configuration. As such, the stent 104 can be biased toward a first configuration while remaining sufficiently flexible to allow the stent 104 to be reconfigured. For example, the stent 104 can be biased toward an expanded state and can be able to be reconfigured and/or transitioned to a collapsed state in response to an applied force. In some implementations, the force can be applied by an actuator or the like that can be manipulated by an operator, can be applied by a sheath, conduit, and/or catheter substantially surrounding the stent 104 in a way that radially compresses the stent (or any other suitable form of compression), and/or can be applied in any other suitable manner. In some implementations, the stent 104 can be a self-expanding stent that is over-expanded (e.g., beyond an extent associated with and/or resulting from self-expansion) by the expansion of the expansion member 101 (e.g., the inflation and/or dilation of a balloon).

In some embodiments, the arrangement of the stent 104 in the expanded state can be such that an outer diameter of the stent 104 is tapered from a larger diameter at or near a distal portion of the stent 104 to a smaller diameter at or near a proximal portion of the stent 104, as shown in FIG. 2. In other embodiments, the stent 104 can have a relatively consistent diameter along a length of the stent 104 (e.g., distal to proximal length), can have a varied diameter with at least one portion and/or section having a larger diameter that at least one other portion and/or section, and/or can have any other suitable size and/or shape (e.g., when in the expanded state). Moreover, the stent 104 in the collapsed or unexpanded state can have a relatively small and relatively constant diameter and/or radial extent.

Although not shown in FIGS. 1 and 2, the stent 104 (e.g., in the expanded state) can form a set of cells and/or can otherwise form a wire frame, mesh, and/or the like. For example, the stent 104 can be formed from a nitinol tube that is laser-cut to form one or more sets of openings along the circumference of the tube. In turn, the remaining structure once expanded can form a wire or wire-like structure with a set of cells corresponding to the laser-cut openings. As described in further detail here with reference to specific embodiments, the cells can have any suitable size, shape, and/or configuration that can result in a desired set of characteristics. For example, in some implementations the stent 104 can have a set of cells having a first size allowing for a first amount of expansion while in other implementations, the stent 104 can have a set of cells having a second size smaller than the first size that allows for a second amount of expansion less than the first amount of expansion. Similarly stated, in some implementations, forming the stent 104 with larger cells can allow the stent 104 to expand to a larger diameter than if the stent 104 had smaller cells. In some implementations, the stent 104 can include any number of sets or subsets of cells that can form, for example, cell segments of the stent 104. For example, in some embodiments, the stent 104 can include a first cell segment (e.g., a distal cell segment) having a subset of cells each with a first size and can include a second cell segment (e.g., a proximal cell segment) having a subset of cells each with a second size, smaller than the first size. In some embodiments, the stent 104 can include more than two cell segments (e.g., three, four, five, six, seven, eight, or more) that can be distributed long a proximal-distal length of the stent 104 (e.g., a longitudinal length and/or in a longitudinal direction along the stent 104).

In some embodiments, the cell segments can be configured to produce and/or result in a desired set of characteristics associated with that cell segment and/or a portion of the stent 104. For example, in some embodiments, a first cell segment can be configured to produce a desired amount of force when expanded to a predetermined size and/or diameter and a second cell segment that can be configured to increase a flexibility of the stent 104 along a portion of the stent 104 allowing the stent to be navigated through tortuous vessels and/or the like. In some implementations, the stent 104 can include alternating cell segments in which a subset of cell segments configured to produce a desired amount of force when expanded a predetermined amount is alternated with a subset of cell segments configured to increase a flexibility of a portion of the stent 104. As such, the stent 104 can be selected, designed, configured, tuned, adapted, etc. to have any suitable characteristic(s) and/or to have sections and/or segments with desired characteristics. In some implementations, the arrangement and/or distribution of cell segments can be based at least in part on one or more characteristics associated with the clot C (e.g., a size, shape, contour, hardness, etc. of the clot C, a degree of occlusion of the vessel V, and/or any other suitable characteristic). In some implementations, the stent 104 can be selected, designed, configured, tuned, adapted, etc. to be over-expanded, for example, in response to a radially outwardly directed force exerted by an expansion member 101 and/or any other suitable component or feature of the system 100.

Although not shown in FIGS. 1 and 2, the stent 104 can include and/or can be coupled to a pusher, cable, actuator, introducer, and/or the like (referred to herein as “pusher”). The pusher, for example, can be included in and/or at least temporarily coupled to the proximal portion of the stent 104 and can be used to advance the stent 104 through the catheter 107 and into the clot C. In some embodiments, the proximal portion of the stent 104 can include one or more struts that at least temporarily couple the stent 104 to the pusher. In some implementations, the pusher can be used as an introducer that defines a lumen configured to receive at least a portion of the stent 104 (e.g., in the collapsed state). In such implementations, the pusher can retain the stent 104 in the collapsed state as the stent 104 is advanced through the catheter 107 and at least partially through the clot C. Once the stent 104 is in a desired position relative to the clot C, the pusher can be retracted and/or disconnected to allow the stent 104 to transition to the expanded state. In other implementations, the pusher can include and/or can function as an actuator that mechanically, electrically, electro-mechanically, and/or thermally actuates the stent 104 (and or the expansion member 101), which in turn, transitions from the collapsed state to the expanded state.

The expansion member 101 of the system 100 can be a member, device, and/or component configured to transition from a first or collapsed state to a second or expanded state to engage and/or contact an inner circumferential wall of the vessel V to, for example, occlude a lumen of the vessel V. In addition, in some instances, transitioning the expansion member 101 to the expanded state can also be used to transition the stent 104 from the collapsed state to the expanded state (and/or an over-expanded state). In some implementations, a distal portion of the expansion member 101 in the expanded state has a first size associated with a diameter of the vessel V, while a proximal portion of the expansion member 101 has a second size smaller than the first size. In some embodiments, the expansion member 101 can include a radiopaque marker allowing for visualization of at least a portion of the expansion member 101 using imaging techniques such as fluoroscopy and/or the like. As shown in FIGS. 1 and 2, the expansion member 101 can be hollow and/or can define at least one opening allowing the expansion member 101 to receive and be advanced along the guidewire 109 (or guidewire catheter).

The expansion member 101 can be any suitable shape, size, and/or configuration. For example, the expansion member 101 can be a balloon configured to transition between a collapsed or uninflated configuration having a first size and an expanded or inflated configuration having a second size greater than the first size. In some embodiments, such a balloon can be a relatively soft, relatively non-compliant, single-lumen balloon that allows for navigation in tortuous arteries and used in the case of relatively good recanalization due to a “soft” clot. In other embodiments, the balloon is a compliant, semi-compliant, or non-compliant double-lumen balloon used, for example, in the case of relatively “hard” clots and/or stenotic atheromasic plaque with insufficient expansion and/or deformation of the stent 104 after deployment.

In other embodiments, the expansion member 101 can be a reconfigurable cap, plug, seal, valve, obturator, and/or the like. For example, in some embodiments, the expansion member 101 is a reconfigurable atraumatic cap configured to be transitioned between a collapsed state and an expanded state. In some implementations, the atraumatic cap can have an umbrella-like configuration in which the cap has a relatively small diameter in the collapsed state and a relatively large diameter (e.g., in the shape of a dome, parabola, cone, etc.) in the expanded configuration. In some embodiments, the system 100 can include two or more expansion members 101 and/or an expansion member with two or more components, portions, features, etc. For example, in some embodiments, the system 100 can include a first expansion member (or a first portion of an expansion member) configured as an inflatable balloon and a second expansion member (or a second portion of an expansion member) configured as a cap, plug, seal, valve, obturator, etc. The expansion members or expansion member portions can be used to perform one or more separate or independent functions and/or can be used to perform one or more combined or collective functions, such as any of the functions described herein. For example, a first expansion member or portion can be a balloon used to expand and/or over-expand the stent 104, while a second expansion member or portion can be an atraumatic cap used to at least partially block or occlude the vessel distal to the clot.

As shown in FIGS. 1 and 2, the expansion member 101 can be advanced into and/or through the stent 104 such that a distal portion of the expansion member 101 is distal to the stent 104 and the clot C. As described in further detail herein, this arrangement of the expansion member 101 can be such that when in the expanded state, the expansion member 101 blocks or occludes the lumen of the vessel V distal to the stent 104 and/or clot C, thereby limiting and/or substantially preventing clot fragments from flowing through the vessel V distal to the expansion member 101. In some implementations, the expansion member 101 in the expanded state can be configured to engage the inner wall of the vessel V such that the application of traction on a proximal end of the expansion member 101 (e.g., outside of the body) pulls the distal portion of the expansion member 101 (e.g., a balloon, cap, plug, etc.) along the inner wall of the vessel V in a proximal direction. As such, the expansion member 101 can scrape and/or push the clot C toward, for example, the catheter 107, which in turn, can be used to aspirate the clot C.

As shown, in some embodiments, the system 100 and/or the distal portion of the stent 104 optionally can include a filter 106 configured to block or filter clot fragments dislodged by the stent 104. The filter 106 can be any suitable shape, size, and/or configuration. For example, in some embodiments, the filter 106 can be formed from graft material used, for example, on stent grafts. In some embodiments, the filter 106 can be a curtain-like membrane on the distal end of the stent 104 (e.g., a portion of the stent 104 without a metallic frame) that is formed from the same material as the expansion member 101 and/or one or more different materials. Such materials can include, for example, polyteirafluoroethylene (PTFE), polyethylene terephthalate (PET), Dacron®, bioabsorbable polymeric materials (such as any of those described above), and/or the like. In some embodiments, the expansion member 101 can include and/or form the filter 106. In some implementations, the system 100 can be used without the optional filter 106. In some implementations, the system 100 can be used without a filter.

The catheter 107 of the system 100 can be used to at least deliver the stent 104 and expansion member 101 through the vessel V to a desired location relative to the clot C. In some embodiments, catheter 107 can be a multi-lumen catheter 107 with a first lumen 118 used to deliver the stent 104 and expansion member 101 and a second lumen 119 used to aspirate the vessel V proximal to the clot C. In some embodiments, the catheter 107 can include and/or define more than the first lumen 118 and the second lumen 119 (e.g., can define three lumens, four lumens, or more). For example, in some implementations, a third lumen of the catheter 107 can be used to deliver one or more probes, sensors, actuators, filters, etc. In some embodiments, the catheter 107 can include and/or form an extended portion configured to be passed through the clot C. As such, the extended portion could function, for example, as a guidewire for the stent 104 and expansion member 101. In some implementations, the extended portion can define a lumen that can allow for at least partial recanalization of the vessel V once the extended portion is advanced through the clot C. For example, the catheter 107 can define an opening proximal to the clot C that is in fluid communication with the lumen of the extended portion of the catheter 107. Thus, a flow of blood can enter the opening and flow through the clot C via the lumen of the extended portion, thereby at least partially perfusing the vessel V distal to the clot C. The stent 104 and the expansion member 101 can then be advanced along the extended portion of the catheter 107 and used to engage and remove the clot C.

While the catheter 107 is shown in FIGS. 1 and 2 as a multi-lumen catheter defining the first lumen 118 and the second lumen 119, in other embodiments, the system 100 can include at least one catheter collectively having at least a first lumen and a second lumen. For example, the system 100 can include a first catheter and a second catheter collectively having a first lumen (e.g., defined by the first catheter) and a second lumen (e.g., defined by the second catheter). Moreover, the first catheter and the second catheter can be coaxial or non-coaxial (e.g., side-by-side), delivered collectively via a delivery catheter or delivered separately and/or independently, similarly sized or configured or independently sized or configured based at least in part on intended usage, and/or the like. Accordingly, while the catheter 107 is particularly shown and described, it should be understood that the system can include any suitable catheter(s) or arrangement of catheter(s).

In use, the catheter 107 can be advanced through the vessel V to a desired position proximal to the clot C where each of the stent 104 and the expansion member 101 can be advanced distally along the guidewire 109 (or guidewire catheter or extended portion of the catheter 107) from the lumen 118 of the catheter 107 and into and/or through the clot C, as indicated by the arrow AA in FIG. 1. In some implementations, the stent 104 and the expansion member 101 can be advanced concurrently. In other implementations, the stent 104 can be advanced prior to advancing the expansion member 101. Moreover, the substantially annular and/or hollow configuration of the stent 104 allows the expansion member 101 to extend through an interior of the stent 104 to place the distal portion distal to the stent 104 and the clot C.

Once advanced relative to the clot C, the stent 104 and the expansion member 101 are then transitioned to the expanded state, as indicated by the arrows BB in FIG. 2. In some implementations, the stent 104 and the expansion member 101 are expanded concurrently such as, for example, when the expansion member 101 is a balloon that is inflated resulting in expansion of the stent. In other implementations, the stent 104 is expanded prior to the expansion member 101. In still other implementations, the expansion member 101 or at least the distal portion thereof (e.g., when the expansion member 101 is an atraumatic cap or the like) is transitioned prior to the stent 104.

The stent 104 being placed in the expanded state results in an outer surface of the stent 104 contacting and/or engaging the clot C (e.g., an interior of the clot C). As such, the stent 104 traps and/or compresses the clot C between the outer surface of the stent 104 and the inner circumferential wall of the vessel V. As described above, the stent 104 can be selected, designed, configured, tuned, adapted, etc. to exert a desired amount of a radially outwardly directed pressure on the clot C. For example, as shown in FIG. 2, the distal portion of the stent 104 in the expanded state has a diameter that is larger than a diameter of a proximal portion of the stent 104. As such, in some instances, the distal portion of the stent 104 can exert a pressure on a distal portion of the clot C that is greater than a pressure exerted by the proximal portion of the stent 104 on a proximal portion of the clot C. In some such instances, the pressure gradient can facilitate movement of the clot C in the proximal direction (e.g., toward the catheter 107), rather than movement of the clot C in the distal direction (e.g., distal to the clot C).

The expansion member 101 being placed in the expanded state results in a distal portion of the expansion member 101 contacting and/or engaging a portion of the inner wall of the vessel V distal to the clot C. As such, the distal portion of the expansion member 101 can form a seal with the inner wall of the vessel V that occludes the lumen of the vessel V and limits and/or substantially prevents clot fragments from flowing distal to the expansion member 101. In some implementations, the distal portion of the expansion member 101 can be moved or scraped along the inner wall of the vessel V in a proximal direction in response to an operator applying a force or traction on a portion of the expansion member 101 (or actuator coupled thereto) disposed outside of the body. In some instances, the proximal movement of the expansion member 101 can help dislodge and/or otherwise move the clot C toward the catheter 107.

With the expansion member 101 and the stent 104 in the expanded state, the vessel V proximal to the distal portion of the expansion member 101 can be aspirated via the catheter 107. For example, in some implementations, a suction or vacuum source can produce a negative pressure in the second lumen 119 of the catheter 107, which in turn, draws blood, clot fragments, and/or some or all of the clot C into the second lumen 119, as indicated by the arrow CC in FIG. 2. In some implementations, the arrangement of the first lumen 118 and the second lumen 119 of the multi-lumen catheter 107 can allow for simultaneous or at least parallel deployment of the stent 104 and expansion member 101 and aspiration of the vessel V. With the distal portion of the expansion member 101 forming a substantial seal with the inner wall of the vessel V, the suction from the aspiration results in a force applied on the clot C operable to draw the clot C into the second lumen 119. In some instances, after aspirating the vessel V and drawing the clot C or at least most of the clot C into the second lumen 119, the expansion member 101 and stent 104 can be transitioned from the expanded state to the collapsed state and can be retracted into the first lumen 118 of the catheter 107. For example, in implementations in which the expansion member 101 is a balloon, the balloon can be deflated to transition to the collapsed or uninflated state. The stent 104 can be transitioned to the collapsed state (e.g., via an actuator or the like) or can be some retracted while in the expanded state or a semi-expanded state and transitioned to the collapsed state as the stent 104 is retracted into the first lumen 118 of the catheter 107. In some instances, aspiration of the vessel V via the second lumen 119 (or via a lumen of a second separate and/or coaxial catheter) can continue as the expansion member 101 and stent 104 are collapsed and retracted into the first lumen 118 of the catheter 107. The continued aspiration can, in turn, draw any additional clot fragments and/or other debris resulting from the transition and/or retraction of the expansion member 101 and stent 104. After providing a desired amount of aspiration and/or after providing a desired degree of reperfusion of the vessel V, the catheter 107 (and/or any additional catheter) can be retracted through the vessel V and out of the body. Thus, the system 100 can be used to dislodge, aspirate, remove, and/or bypass the clot to restore blood flow through the vessel V.

Although not shown in FIGS. 1 and 2, in some embodiments, the system 100 can optionally include one or more probes, devices, and/or components configured to sense one or more characteristics associated with blood flow through the vessel V, pressure exerted by the stent 104, and/or any other suitable characteristic. For example, in some embodiments, the system 100 can optionally include one or more flow sensors configured to sense a flow of blood through the vessel V by determining a temperature distribution in a portion of the vessel V (e.g., in response to a heat source providing heat). In other embodiments, the system 100 can include one or more optical interferometric temperature sensors to sense and/or measure, for example, thermally induced changes in the wavelength of light produced by optical fiber light source.

In some embodiments, a force sensing resistor and/or strain gauge can be integrated with, embedded on, and/or otherwise coupled to an outer surface of the stent 104 or expansion member 101 and configured to sense a force or pressure exerted by the stent 104 on the clot C or exerted by the expansion member 101 on the walls of the vessel V, respectively. In some implementations, such a sensor can be powered externally via one or more electrical wires passed through the catheter 107 or configured to transmit or broadcast wirelessly (e.g., via low power interrogation performed at short range, for example, by holding a reader or the like up to the head or neck of the patient). In some implementations, data associated with the pressure exerted on the walls of the vessel V and/or clot C can, for example, prevent overstressing of constricted, potentially atherosclerotic vessels, which could lead to second degree hemorrhage, and/or can allow an operator to determine whether the clot C is a “soft” or “hard” clot.

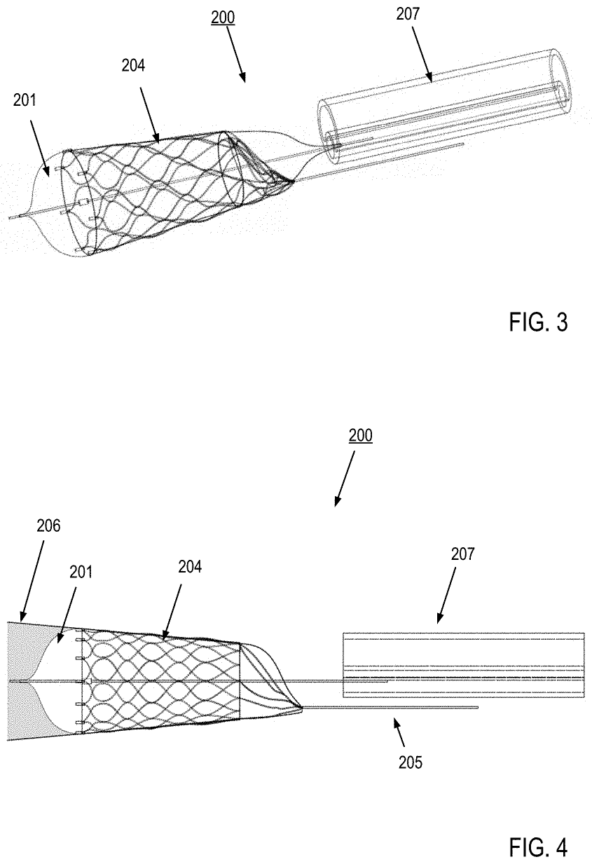

FIGS. 3-14 illustrate a reperfusion system and/or device 200 according to an embodiment. In some implementations, an introducer system is placed to the femoral artery and a guidewire (not shown) is advanced through the arterial access site of the body. A delivery sheath is advanced over the guidewire until the tip of the catheter is placed to the desired location. The temporary endovascular clot bypass system and/or device 200 is loaded to a loader and flushed with a saline solution to reduce and/or substantially eliminate the risk of residual air bubbles which might cause air-embolization. The reperfusion system and/or device 200—referred to herein as “device 200”—is connected to a detachable pusher cable 205 to advance and retrieve the device 200 (and/or one or more portions thereof) at a desired location and time. The guidewire is advanced through cerebral arteries passing inside of the target blood clot.

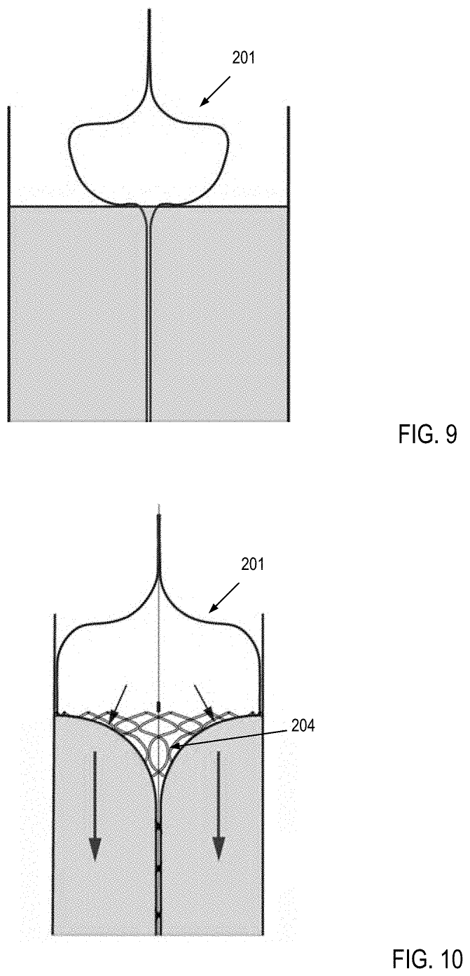

The device 200 includes a stent device 204 that can be double diameter (e.g., expandable between a collapsed configuration and/or state having a first diameter and an expanded configuration and/or state having a second diameter larger than the first diameter), made from and/or including a shape memory material, and/or formed into a metallic braided laser-cut mesh. The stent 204 is navigated within a multi-lumen catheter (e.g., a double lumen microcatheter) 207 and positioned through the blood clot (see e.g., FIG. 9). For example, the stent 204 can have any number of struts or the like connecting the stent 204 to the pusher cable 205, which in turn, can be used to navigate, advance, and/or retract at least the stent 204 through a lumen of the multi-lumen catheter 207. While the pusher cable 205 is shown outside of the multi-lumen catheter 207 in FIGS. 3 and 4, it should be understood that the pusher cable 205 can extend through any suitable lumen of the multi-lumen catheter 207.

Once the stent 204 is deployed to the target area, the stent 204 can be expanded (e.g., from a collapsed configuration and/or state to an expanded configuration and/or state) to embed the clot within the stent 204. Once the clot is entrapped within the temporary endovascular clot bypass stent 204, an expansion member can be deployed. In this embodiment, the expansion member is an inflatable balloon 201 (see e.g., FIGS. 3-5, 9-12, and 14).

In some implementations, the balloon 201 is a double diameter (e.g., expandable between a collapsed configuration and/or state having a first diameter and an expanded configuration and/or state having a second diameter larger than the first diameter), relatively soft, relatively non-compliant, single-lumen balloon 201 that can be relatively easy to navigate in tortuous arteries and used in the case of relatively good recanalization due to a “soft” clot. In some instances, relatively good recanalization due to a soft clot can be achieved without deploying and/or inflating the balloon 201.

In some implementations, the balloon 201 is a compliant, semi-compliant, or non-compliant double-lumen balloon 201 used, for example, in the case of relatively “hard” clots and/or stenotic atheromasic plaque with insufficient expansion and/or deformation of the stent 204 after deployment.

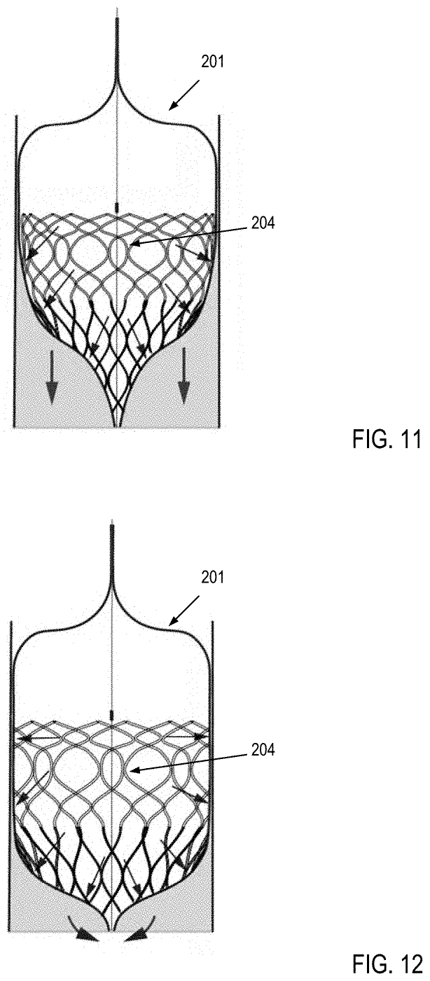

In some implementations, the balloon 201 is configured to be advanced over the guidewire (or a separately deployed guidewire). The balloon 201 can be navigated along the guidewire into and/or through the stent 204, see e.g., FIG. 9, and to the entrapped clot area through a guidewire lumen 202 formed by and/or included in the stent. Once in the entrapped clot area, the balloon 201 can be inflated, expanded, and/or otherwise transitioned from a deflated state to an inflated state, FIGS. 10-12. The balloon 201 is configured such that, during deployment (e.g., when the balloon 201 is in the inflated state), the balloon 201 has a larger distal diameter than proximal diameter, which can allow the balloon 201 to trap the clot between an outer surface of the stent 204 and the artery wall, as shown in FIG. 11. Moreover, the balloon 201 extends through the expanded stent 204 such that a distal portion of the balloon 201 extends beyond (e.g., distal to) the stent 204. As such, an outer surface at or along the distal portion of the balloon 201 can contact and/or engage the artery wall (or the wall of any other vessel), thereby forming at least a temporarily seal.

Trapping of the clot between the artery and stent 204 outer surface can be initiated by the inflation of the balloon 201 pushing the clot backward to eliminate the risk of distal embolization from the non-occluded distal part of the artery, as shown in FIGS. 10-12.

The balloon 201 includes a radiopaque marker 203 to show the location of the stent 204 and/or balloon 201. In some implementations, the balloon surface pressurizes the entrapped clot between artery wall and the stent 204 after inflation of the balloon 201. In some implementations, the inflation of the balloon 201 can expand the stent 204 to open the lumen resulting in simultaneous recanalization. The balloon 201 is then deflated to result in and/or allow distal perfusion.

As shown in FIGS. 3, 7, 13, and 14, the device 200 can include a free distal prolongation of the stent-graft, a curtain-like membrane on the distal end of the stent 204 without a metallic frame, which is placed as a flow driven filter 206 to block clot particles from flowing into the vessel lumen and preventing distal embolization by blocking the distal side of the artery near the distal part of the inflated balloon 201. In some instances, the distal part of the inflated balloon 201 can press the filter 206 against the vessel walls, thereby blocking the vessel lumen distal to the stent 204. In some implementations, the filter 206 can be a sleeve, membrane, and/or the like that is deployed over at least a portion of the stent 204 (e.g., when the stent 204 is in or at a clot location and at least partially collapsed). In such implementations, the expansion of the stent 204 can be operable to couple the filter 206 to the stent 204. In other implementations, the filter 206 can be integrally formed with the stent 204 and/or otherwise pre-coupled to the stent 204 prior to use (e.g., during manufacturing). For example, the filter 206 can be coupled to at least a portion of an external surface of the stent 204 or an internal surface of the stent 204.

In some implementations, aspiration during removal of the stent 204 can aspirate all or substantially all clots trapped between the vessel wall and the external surface of the stent 204. Aspiration of the vessel can be performed via the multi-lumen catheter 207. For example, in some implementations, a first lumen of the catheter 207 can be used to deliver and/or provide the stent 204, the balloon 201, and/or the filter 206, while a second lumen of the catheter 207 can be used for aspiration (e.g., suction). After aspiration (e.g., after providing flow restoration), the balloon 201 is deflated and the deflated balloon 201 and then the stent 204 (e.g., in the collapsed state or a semi-collapsed state) can be pulled back into the multi-lumen catheter 207 (e.g., the first lumen of the multi-lumen catheter 207).

In some embodiments, the stent 204 is made from a shape-memory alloy or material like Nitinol® and/or is similarly designed for mounting on a guidewire and/or in the guidewire lumen 202. The balloon 201 is also designed for mounting on a guidewire and/or in the guidewire lumen 202. The stent 204 and the balloon 201 can be advanced over the guidewire to function together to open a plugged or occluded area in the body when released at the desired location. The balloon 201 can be used for expansion of the stent 204, where a distal diameter of the balloon 201 is larger than a proximal diameter to trap the clot between stent 204 and artery wall. The stent 204 and the balloon 201 can be advanced over the guidewire concurrently (e.g., together), sequentially (e.g., one after the other), and/or otherwise independently (e.g., one without the other).

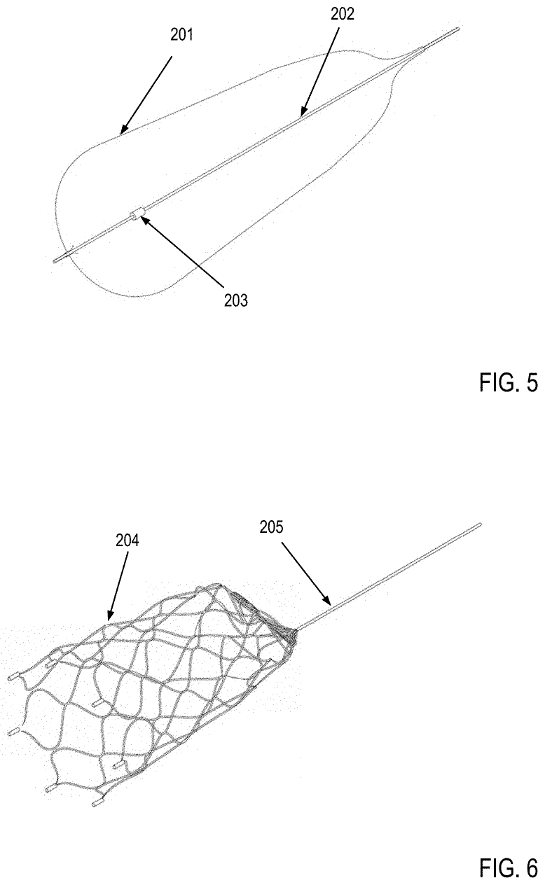

The free distal prolongation of the stent-graft (also referred to as the filter 206) can be a curtain-like membrane on the distal end of the stent 204 without a metallic frame and used as a flow driven filter. The filter 206 can be formed from either the same material as the stent 204 and/or balloon 201 or one or more different materials. Such materials can include, for example, polytetrafluoroethylene (PTFE), polyethylene terephthalate (PET), Dacron®, bioabsorbable polymeric materials (such as any of those described above), and/or the like. The filter 206 can be used to block clot particles from coming into the vessel lumen and preventing distal embolization. The balloon 201 and/or any other suitable portion of the device 200 includes a radiopaque marker 203 from material like gold, platinum, iridium, tantalum or similar (such as any of the radiopaque materials described above) to show the location of the stent 204 on the balloon surface (FIG. 5).

In some implementations, a delivery system can optionally include a detachable pusher cable 205, see e.g., FIGS. 4 and 6, used to advance and/or retrieve the stent 204 and/or other portions of the device 200 (e.g., the balloon 201, filter 206, and/or the like). The delivery system can also optionally include a loader, a delivery sheath, and the multi-lumen catheter (e.g., a double lumen microcatheter) 207. The multi-lumen catheter 207 can be used to advance the self-expandable stent 204 and/or the balloon 201 over the guidewire within one lumen and the other lumen can be used for continuous aspiration. Alternatively, the catheter 207 can be used to advance the stent 204 via a first lumen, to advance the balloon 201 via a second lumen, and to provide aspiration via the first lumen, the second lumen, and/or a separate third lumen.

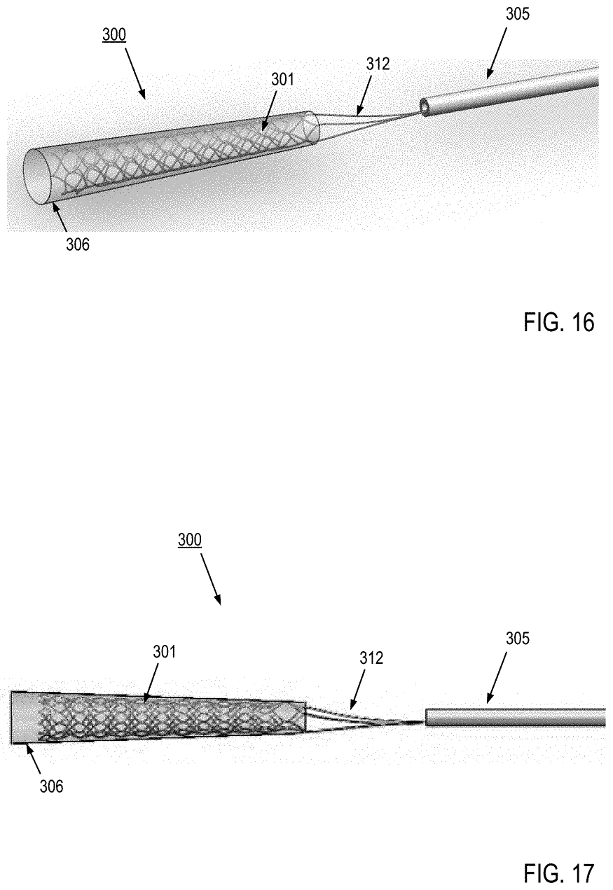

FIGS. 16-20 illustrate at least a portion of a reperfusion system and/or device 300 (“system 300”) according to another embodiment. More particularly, the system 300 includes at least a stent 301, a pusher 305, and a filter 306. The stent 304, pusher 305, and filter 306 (and/or aspects or portions thereof) can be similar to and/or substantially the same as the stent 104, pusher, and filter 106, respectively, described above with reference to FIGS. 1 and 2. Accordingly, such aspects and/or portions are not described in further detail herein.

FIGS. 16 and 17 are a perspective view and a side view, respectively, showing the stent 304 in an expanded state with a proximal portion thereof coupled to the pusher 305 via a set of struts 312 and with the filter 306 disposed about an outer surface of the stent 304. The filter 306 can be any suitable shape, size, and/or configuration. In this embodiment, the filter 306 is, for example, a relatively thin membrane or the like that forms a sleeve extending over at least a portion of the stent 304. The filter 306 can be formed from graft material used, for example, on stent grafts such as Dacron® and/or the like. In other embodiments, the filter 306 can be formed from a polymer such as PTFE, PET, and/or any other suitable biocompatible material. As described above with reference to the filter 106, the filter 306 shown in FIGS. 16 and 17 can be used to block or filter clot fragments dislodged by the stent 304. In some implementations, the filter 306 can be used in conjunction with an expansion member such as the expansion members 101 and 201 or can be used as an alternative to and/or instead of an expansion member.

While the filter 306 is shown in FIGS. 16 and 17 as being disposed about stent 304 in its entirety or substantially in its entirety, in other embodiments, the system 300 can include one or more filters 306 disposed about a portion of the stent 304. For example, as described above with reference to the filter 106, in some embodiments, the filter 306 can be disposed about and/or coupled to a distal end portion of the stent 304. In some embodiments, the system 300 can include two filters 306, with a first filter disposed at or near the distal end portion of the stent 304 and a second filter disposed at or near a proximal end portion of the stent 304.

In some implementations, the filter 306 can have and/or can perform multi-functions. For example, as shown in FIGS. 16 and 17, the filter 306 can surround and/or extend along a length of the stent 304, which in turn, can be operable in retaining the stent 304 in a collapsed or at least partially collapsed state. In such implementations, after the stent 304 is in a desired position relative to a clot in a vessel, the filter 306 can be advanced in a distal direction relative to the stent 304 and can, for example, collect, gather, bunch, etc. at or near a distal end of the stent 304 (e.g., pulled from the proximal end of the stent 304 to or toward the distal end, thereby uncovering or exposing at least a portion of the stent 304). In some implementation, the filter 306 can be advanced in the distal direction relative to the stent 304 and can, for example, invert or turn inside-out, with a portion of the filter 306 remaining attached to the distal end of the stent 304. With the filter 306 no longer disposed about the stent 304 (or a majority of the stent 304), the stent 304 can be allowed to transition from the collapsed or at least partially collapsed state to the expanded state to contact and/or engage the clot through which the stent 304 at least partially extends.

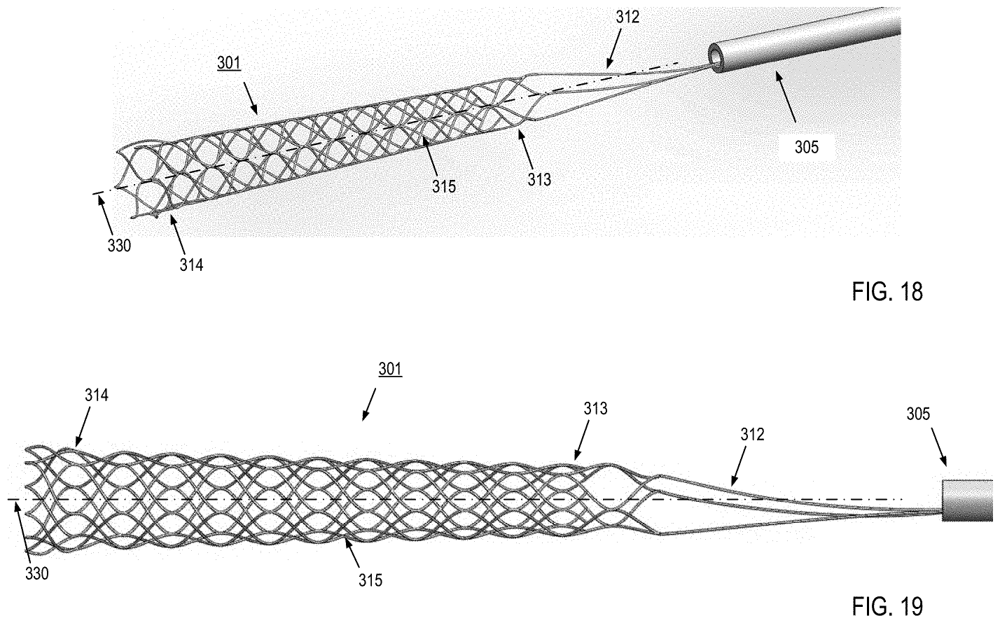

FIGS. 18 and 19 are a perspective view and a side view, respectively, showing the stent 304 in an expanded state and the pusher 305 without the filter 306 disposed about the stent 304. The pusher 305 can be included in and/or at least temporarily coupled to a proximal end portion 313 of the stent 304 and can be used to advance the stent 304 through a catheter (e.g., the multi-lumen catheters 107 and/or 207 described above) and into the clot C. For example, the proximal end portion 313 of the stent 304 includes a set of struts 312 that at least temporarily couple the stent 304 to the pusher 305. In some implementations, the pusher 305 can be used as an introducer that defines a lumen configured to receive at least a portion of the stent 304 (e.g., in a collapsed state). In such implementations, the pusher 305 can retain the stent 304 in the collapsed state as the stent 304 is advanced through the catheter and at least partially through the clot. Once the stent 304 is in a desired position relative to the clot, the pusher 305 can be retracted and/or disconnected to allow the stent 304 to transition to the expanded state. In other implementations, the pusher 305 can include and/or can function as an actuator that mechanically, electrically, electro-mechanically, and/or thermally actuates the stent 304 (and/or an expansion member), which in turn, transitions from the collapsed state to the expanded state. Accordingly, the pusher 305 can be used to facilitate the positioning and/or transitioning of the stent 304 relative to the clot.

In this embodiment, the stent 304 can have a substantially annular shape (e.g., a hollow shell in the shape of a cylinder) with an open distal end and an open proximal end, that can allow the stent 304 to be advanced over and/or along a guidewire or guidewire catheter (not shown) and/or can allow at least a portion of the system 300 (e.g., an expansion member) and/or any other suitable member to be disposed in or at least partially advanced through the stent 304 from a proximal position to a distal position (or vice versa).

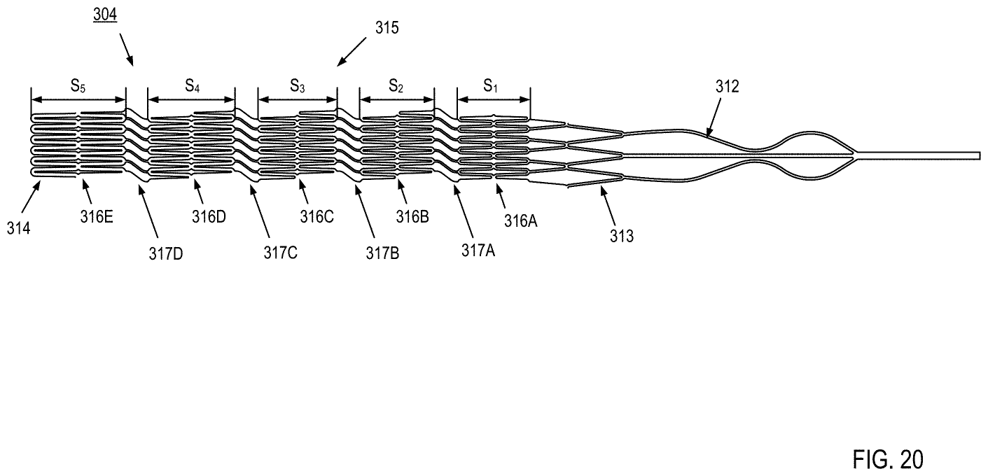

In the embodiment shown in FIGS. 16-20, the stent 304 is an expandable frame or structure configured to transition between a collapsed configuration and/or state (not shown) having a first size and an expanded configuration and/or state having a second size larger than the first size. As described above with reference to the stent 104, the arrangement of the stent 304 in the collapsed or unexpanded state can allow at least a portion of the stent 304 to be inserted in and/or through a clot in a vessel, while the arrangement of the stent 304 in the expanded state can be allow for, result in, and/or otherwise facilitate a desired engagement with and/or entrapment of the clot in the vessel. In this embodiment, the stent 304 is a self-expanding structure formed from a superelastic and/or shape-memory material such as a nickel-titanium alloy (e.g., Nitinol® and/or the like). For example, the stent 304 can be laser cut from a sheet or tube of Nitinol® (FIG. 20) and expanded and/or heat-set into a desired shape, size, and/or configuration.

As shown in FIGS. 18 and 19, the arrangement of the stent 304 in the expanded state can be such that an outer diameter of the stent 304 is tapered from a larger diameter at or near a distal end portion 314 of the stent 304 to a smaller diameter at or near a proximal end portion 313 of the stent 304. The proximal end portion 313 of the stent 304 includes a set of struts 312 that at least temporarily couple the stent 304 to the pusher 305. While the set of struts 312 is shown as including three struts, in other embodiments, the stent 304 can include fewer than three struts (e.g., two struts or one strut) or more than three struts (e.g., four struts, five struts, six struts, seven struts, eight struts, or more).

The stent 304 is and/or includes a wire frame, mesh, and/or the like that forms a set of cells 315. As described in further detail here with reference to specific embodiments, the cells 315 can have any suitable size, shape, and/or configuration that can result in a desired set of characteristics. For example, the expanded stent 304 is shown as including a set of wave-shaped structures or wires that define each cell 315 between the intersections of two or more of the wave-shaped wires. In some embodiments, the cells 315 can be substantially uniform in size, shape, and/or distribution along the stent 304. In other embodiments, the size and/or shape of the cells 315 can be varied along the length of the stent 304. For example, the stent 304 is arranged such that the cells or cell segments (e.g., a subset of cells distributed circumferentially at a given positional along a central axis 330 of the stent 304, as shown in FIGS. 18 and 19) increase in size from a first size at the proximal end portion 313 of the stent 304 to a second size at the distal end portion 314 of the stent 304. In some implementations, varying (e.g., increasing) the cell size along the central axis 330 can result in the expanded stent 304 having a larger diameter at the distal end portion 314 and a smaller diameter at the proximal end portion 313, as described above.

In some embodiments, the cells in each cell segment can be configured to produce and/or result in a desired set of characteristics associated with that cell segment and/or a portion of the stent 304 corresponding to a position along the central axis associated with that cell segment. In some implementations, the desired set of characteristics can be substantially uniform for each cell segment and/or for each cell of the stent 304. In other implementations, the desired set of characteristics associated with one or more cell segments can be different than the desired set of characteristics associated with one or more other cell segments. In some implementations, each cell segment can have and/or can be associated with a unique set of characteristics. Such an arrangement, for example, can allow the stent 304 to have a set of characteristics that can be tailored to and/or at least partially based on the clot to be bypassed or removed and/or the vessel at least partially occluded by the clot to be bypassed or removed.

For example, FIG. 20 is a flat pattern illustration of the stent 304 and showing the set of cells 315 laser-cut into a Nitinol® tube prior to the tube being heat-set into the shape of the stent 304 (e.g., in the expanded state shown in FIGS. 18 and 19). The stent 304 includes alternating cell segments in which a first subset of cell segments configured to produce a desired amount of force when expanded a predetermined amount is alternated with a second subset of cell segments configured to increase a flexibility of a portion of the stent 304. Specifically, the first subset of cell segments includes a first cell segment 316A having a first size S₁, a second cell segment 316B having a second size S₂, a third cell segment 316C having a third size S₃, a fourth cell segment 316D having a fourth size S₄, and a fifth cell segment 316E having a fifth size S₅. In this embodiment, the sizes S₁-S₅ are successively larger, which can result in the increasing diameter of the stent 304 in the expanded state from the smaller diameter at the proximal end portion 313 to the larger diameter at the distal end portion 314. In some embodiments, for example, the size S₅ of the fifth cell segment 316E can be about 1.0 millimeters (mm) larger than the size S₁ of the first cell segment 316A. In other embodiments, the sizes of the cell segments can vary by any suitable amount (e.g., less than 1.0 mm between the smallest size (S₁) and the largest size (S₅) or more than 1.0 mm between the smallest size (S₁) and the largest size (S₅)).

As shown, the second subset of cell segments includes a sixth cell segment 317A disposed between the first cell segment 316A and the second cell segment 316B, a seventh cell segment 317B disposed between the second cell segment 316B and the third cell segment 316C, an eighth cell segment 317C disposed between the third cell segment 316C and the fourth cell segment 316D, and a ninth cell segment 317D disposed between the fourth cell segment 316D and the fifth cell segment 316E. As described above with reference to the sizes S₁-S₅, a size of the cell segments 317A-317D can also successively increase. In other embodiments, a size of each cell segment 317A-317D is substantially the same size.

As described above, each cell segment 316A-316E in the first subset of cell segment is configured to correspond to a specific size and/or diameter of the stent 304 in the expanded configuration. In instances in which a thickness of the clot is substantially constant or uniform (e.g., resulting in a substantially uniform narrowing of a diameter of a lumen of the vessel), the different sizes and/or diameters of the stent 304 can result in a corresponding difference in pressure exerted by the stent 304 at a position along the central axis 330 corresponding to each cell segment 316A-316E. For example, in some implementations, an amount of pressure exerted by the outer surface of the stent 304 on the clot can be proportional to a diameter of the stent 304, which in turn, is at least partially dependent on a size of the cell segments 316A-316E. In some instances, an amount of force or pressure exerted by one or more portions of the expanded stent 304 on the clot and/or vessel walls can be selected, controlled, and/or tuned based at least in part on one or more characteristics associated with the clot (e.g., a size, shape, contour, hardness, etc. of the clot, a degree of occlusion of the vessel, and/or any other suitable characteristic).

As described above, each cell segment 317A-317D in the second subset of cell segments is configured to increase a flexibility of at least a portion of the stent 304 along the central axis 330. In some implementations, the size and/or configuration of each cell segment 317A-317D can result in the expanded stent 304 having a substantially uniform flexibility along the central axis 330 between the proximal end portion 313 and the distal end portion 314. In other implementations, each cell segment 317A-317D can be sized and/or configured to result in the expanded stent 304 having one or more localized portions along the central axis 330 having an increased flexibility or a decreased flexibility. In some instances, selectively controlling and/or tuning the manufacturing, and/or characteristics of the stent 304 to result in a desired amount of flexibility of the stent 304 along the central axis 330 can allow the stent 304 to be navigated through relatively small and/or tortuous vessels such as cerebral arteries and/or the like.

While the size of the first subset of cell segments 316A-316E and of the second subset of cell segments 317A-317D is described above as being varied and/or selected to result in one or more desired characteristics of the stent 304, it should be understand that any other feature of the cells segments can be varied and/or selected to result in one or more desired characteristics. For example, in some implementations, each cell in a first cell segment can have a first shape and each cell in a second segment can have a second shape different from the first shape. In such implementations, the first and second shapes can differ in such a way that results in different strengths, rigidities, flexibilities, abilities and/or extents of expansion, etc. of corresponding portions of the stent.

While the stent 304 is described above as being a self-expanding stent that transitions from the collapsed state to an expanded state (e.g., as a result of being biased to or toward the expanded state) in some implementations, the stent 304 can be self-expanding from the collapsed state to the expanded state, and then can be over-expanded, for example, by an expansion member such as an inflatable balloon and/or the like. For example, an expansion member such as an inflatable balloon can be advanced through the stent 304 and inflated to facilitate or aid the expansion of the stent 304 to the expanded state and/or to “over-expand” the stent 304 to an over-expanded state. In some implementations, the expansion member can be used to expand the stent 304 beyond an extent associated with the self-expansion of the stent 304. The over-expansion of the stent 304 can allow for a desired amount of contact or engagement with a surface of a clot; a desired amount of pressure exerted on at least a portion of the surface of the clot; a staged, gradual, and/or controlled engagement of the clot; and/or the like. Moreover, the expansion member in the expanded state (or at least a distal portion thereof) can engage and/or contact an inner wall of the vessel to at least temporarily block or occlude the vessel distal to a clot, as described in detail above with reference to the expansion members 101 and/or 201.

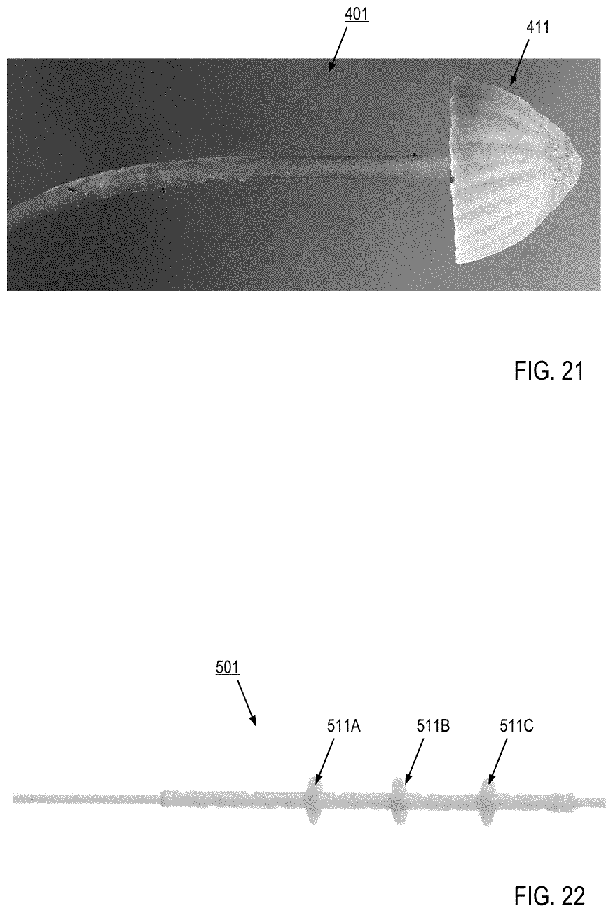

FIG. 21 is a side view illustration of an expansion member 401 according to an embodiment, which can be included in and/or used with any of the systems 100, 200, and/or 300 described above. While the expansion member 201 is described above as being a balloon that is transitioned between an uninflated state and an inflated state, in the embodiment shown in FIG. 21, the expansion member 401 is a relatively small tube, catheter, rod, guidewire, etc. that includes and/or is coupled to a reconfigurable atraumatic distal cap 411 (“distal cap 411”). The expansion member 401 and/or at least the distal cap 411, for example, can be configured to transition between a collapsed state and an expanded state in which the distal cap 411 has and/or forms a mushroom-cap or umbrella-like shape (e.g., a parabolic cross-sectional shape). As such, the distal cap 411 can have a relatively small diameter when in the collapsed state and can be expanded in a radial direction to result in a larger diameter based at least in part on an inner diameter of the vessel in which the expansion member 401 is deployed.

The expansion member 401 can be actuated in any suitable manner to transition the distal cap 411 between the collapsed state and the expanded state. For example, in some embodiments, the expansion member 401 can be mechanically actuated by advancing or retracting a slider, pusher, collar, etc. (e.g., similar to the opening and/or closing of an umbrella). In other embodiments, the distal cap 411 can be electrically and/or electromagnetically actuated. For example, in some implementations, the distal cap 411 can include a metal (e.g., Nitinol®) or otherwise magnetic frame that can be covered with a biocompatible material such as Dacron® and/or any other suitable material described above. In some instances, a central portion of the expansion member 401 can be electromagnetically energized, which in turn, attracts the distal cap 411 toward the central portion, thereby placing the expansion member 401 in the collapsed state. Conversely, after the expansion member 401 is placed in a desired position relative to a stent and/or clot, the central portion of the expansion member 401 can be de-energized, which in turn, allows the distal cap 411 to transition to the expanded state (e.g., the metal and/or magnetic frame of the distal cap 411 is self-expanding). Accordingly, when in the expanded state, at least a portion of the distal cap 411 contacts and/or engages an inner wall of the vessel distal to the clot to limit and/or substantially prevent clot fragments, microemboli, and/or the like from flowing distal to the distal cap 411, as described in detail above with reference to the expansion members 101 and/or 201.

In some implementations, the arrangement of the distal cap 411 of the expansion member 401 can allow the distal cap 411 to be moved or scraped along the inner wall of the vessel in a proximal direction in response to an operator applying a force or traction on a portion of the expansion member 401 (or actuator coupled thereto) disposed outside of the body. In some instances, the proximal movement of the expansion member 401 can help dislodge and/or otherwise move the clot toward a catheter, thereby facilitating aspiration. In some implementations, after aspiration of the clot, the expansion member 401 can be retracted into a lumen of a delivery catheter (e.g., the catheter 107 and/or 207) and the arrangement of the distal cap 411 can allow the distal cap 411 to invert or evert for retraction into the catheter.

FIG. 22 is a side perspective view of an expansion member 501 according to another embodiment. While the expansion member 401 is described above with reference to FIG. 21 as having the distal cap 411, in the embodiment shown in FIG. 22, the expansion member 501 is a relatively small tube, catheter, rod, guidewire, etc. that includes and/or is coupled to a set of caps, plates, rings, discs, etc. (referred to herein as “disc(s)”). For example, in some implementations, the expansion member 501 can include one or more expandable braided discs that can be transitioned between a collapsed state (e.g., having a first diameter) and an expanded state (e.g., having a second diameter greater than the first diameter) via an actuator, pull wire, and/or the like. In the embodiment shown in FIG. 22, the expansion member 501 includes a first or proximal disc 511A, a second or medial disc 511B, and a third or distal disc 511C. In some implementations, the discs 511A, 511B, and 511C can be structurally similar to the distal cap 411 described above. As such, each of the discs 511A-511C can be transitioned from a collapsed state to an expanded state to engage and/or contact a portion of an inner wall of a vessel and/or a portion of a clot in the vessel. For example, in some implementations, the expansion member 501 can include a sheath or the like that can surround the discs 511A-511C to maintain the discs 511A-511C in the collapsed state and once the expansion member 501 is in a desired position relative to the clot, the sheath can be retracted to allow the discs 511A-511C to transition to the expanded state. In other implementations, each disc 511A-511C can be, for example, an inflatable disc similar to the balloon configured of the expansion member 201.

The discs 511A-511C can be configured to collectively transition between the collapsed state to the expanded state (e.g., concurrently, in parallel processes, and/or in response to a single input or actuation) or to independently transition. In some implementations, independently transitioning the discs 511A-511C from the collapsed state can allow the distal most disc 511C to be transitioned to the expanded state first, thereby occluding or sealing a lumen of the vessel distal to the clot. The medial disc 511B and the proximal disc 511A can then be transitioned (or not transitioned) depending on need. In some implementations, the arrangement of the discs 511A-511C can allow for retrograde traction of clot material towards a source of suction (e.g., a multi-lumen catheter). In some instances, selectively and independently controlling each disc 511A-511C can also allow for aspiration of the clot in portions, which may reduce an amount of a suction force used for aspiration.

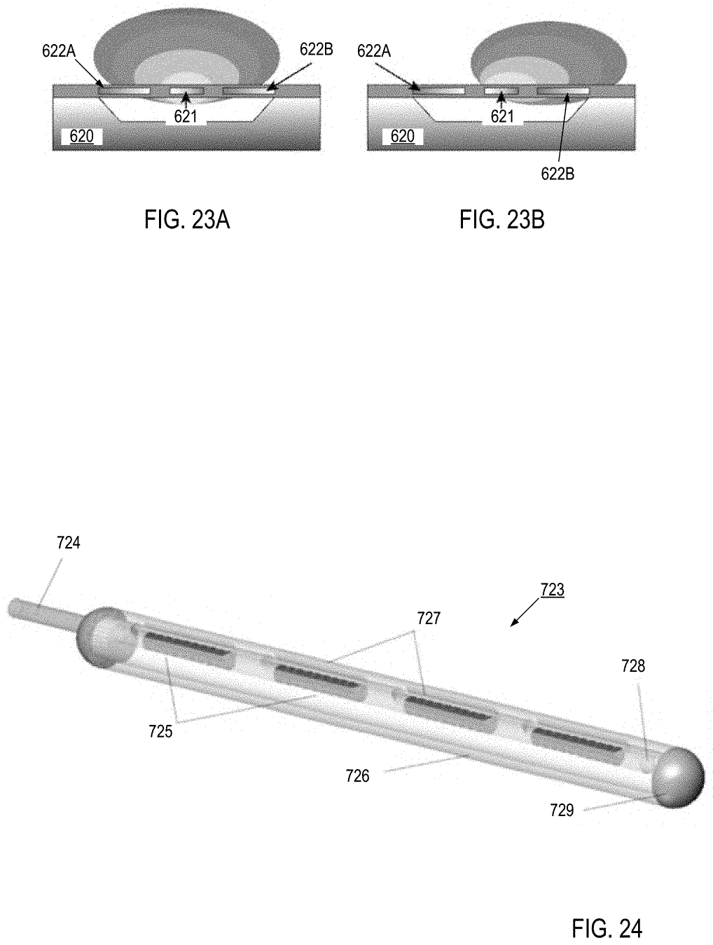

FIGS. 23A and 23B are schematic illustrations of a probe 620 that can be used to determine and/or sense a flow of blood through a vessel, according to an embodiment. For example, the probe 620 can include a heat source 621 and a set of temperature sensors. More particularly, the probe 620 can include a first temperature sensor 622A that is positioned proximal to the heat source 621 and a second temperature sensor 622B that is positioned distal to the heat source 621. In some implementations, for example, the temperatures sensors 622A and 622B can be thermopiles configured to convert thermal energy into electric energy and/or any other suitable temperature sensor. The arrangement of the temperature sensors 622A and 622B relative to the heat source 621 allow for an evaluation of flow through the vessel based on a sensed distribution of thermal energy from the heat source 621.

For example, FIG. 23A illustrates a temperature distribution in the vessel that is associated with and/or indicative of substantially no flow through the vessel. As shown, the temperature distribution is substantially equal and/or uniform between the proximal temperature sensor 622A and the distal temperature sensor 622B. In contrast, FIG. 23B illustrates a temperature distribution in the vessel that is associated with and/or indicative of a flow of blood through the vessel. As shown, the temperature distribution is directed toward the distal temperature sensor 622B rather than the proximal temperature sensor 622A. More particularly, thermal energy from the heat source 621 is at least partially absorbed by blood flowing past the heat source 621, which is sensed by the distal temperature sensor 622B as the blood flows past the distal temperature sensor 622B. Accordingly, with the proximal temperature sensor 622A being upstream of the heat source 621 (e.g., when the probe 620 is disposed in an artery), the temperature distribution is directed, skewed, biased, etc. in a distal direction relative to the heat source 621.

Although not shown in FIGS. 23A and 23B, the probe 620 can be advanced to a desired position within a vessel in any suitable manner. For example, in some implementations, the probe 620 can be delivered via a separate and/or independent delivery catheter. In other implementations, the probe 620 can be delivered via a lumen of a multi-lumen catheter such as the multi-lumen catheters 107 and/or 207 described above. In some implementations, the probe 620 can be embedded and/or integrated into one or more devices (e.g., a stent, expansion member, guidewire, delivery catheter, etc.).

The heat source 621 can be energized to release thermal energy in any suitable manner. In some implementations, for example, a wire or other electrical connection can extend through a lumen of a catheter (e.g., a lumen of a multi-lumen catheter) and connect, at a distal end, to the heat source 621. The proximal end of the wire or other electrical connection can, for example, be disposed outside of the body and connected to a power source configured to provide electric power along the wire to the heat source 621, which in turn, releases the thermal energy. In other implementations, the heat source 621 can be energized wirelessly (e.g., via induction), chemically (e.g., as a result of contact with blood or other fluid, or the like), and/or in any other suitable manner.

FIG. 24 is a schematic illustration of a temperature sensor 723 that can be used to determine and/or sense a flow of blood through a vessel, according to another embodiment. In this embodiment, the temperature sensor is, for example, an optical interferometric temperature sensor, which includes a nylon sleeve 726 with an optical fiber light source 724 coupled to a proximal end of the sleeve 726 and configured to produce a beam of light and an adhesive cap 729 coupled to a distal end of the sleeve 726. The temperature sensor 723 includes a set of fiber Bragg gratings (FBG) 725 disposed in the sleeve 726 and configured to reflect a desired wavelength of light, which in turn, is allowed to pass through a set of holes 727 in the sleeve 726. The temperature sensor 723 further includes an index matching gel 728 disposed within the distal end of the sleeve 726 and configured to approximate an index of refraction associated with the light through the sensor 723. In this manner, the sensor 723 can be positioned within a vessel and used to sense thermally-induced changes in the wavelength of light produced by optical fiber light source 724 as a result of blood flow past the sensor 723 (e.g., a lack of flow in the vessel would result in less thermally-induced change in the wavelength, while a flow of blood through the vessel would result in a greater thermally-induced change in the wavelength).

As described above with reference to the probe 620, the temperature sensor 723 can be advanced to a desired location in the vessel in any suitable manner. For example, the temperature sensor 723 can be delivered via a separate or independent delivery catheter, via a lumen of a multi-lumen catheter (e.g., separate from a lumen that delivers a stent and expansion member, and separate from a lumen that provides aspiration), and/or via any other suitable device. In some embodiments, the temperature sensor 723 can be embedded on and/or integrated with a stent, expansion member, guidewire, catheter, and/or the like. Moreover, the temperature sensor 723 and/or components thereof can be energized in any suitable manner such as those described above with reference to the heat source 621.

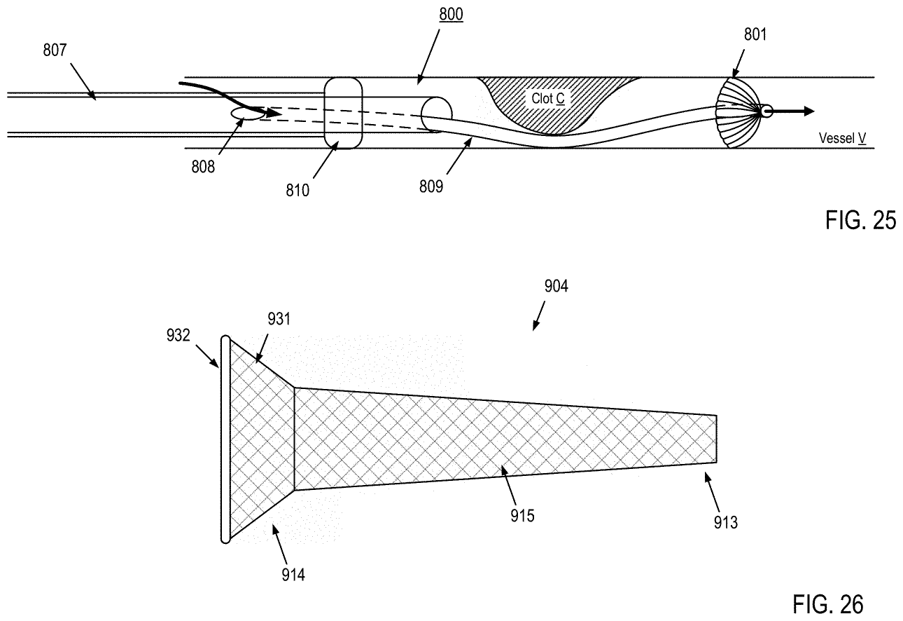

FIG. 25 is a schematic illustration of at least a portion of a reperfusion system and/or device 800 (“system 800”) according to an embodiment. Aspects and/or portions of the system 800 can be similar to and/or substantially the same as the systems 100, 200, and/or 300 described in detail above. Accordingly, such aspects and/or portions of the system 800 are not described in further detail herein.

As shown, the system 800 and/or portion thereof includes at least an expansion member 801, a catheter 807, a guidewire catheter 809, and a proximal balloon 810. The system 800 can also include a stent (not shown) that can be similar in at least form and/or function to any of the stents described herein. As described in detail above, a first lumen of the catheter 807 can be configured to deliver at least a stent and the expansion member 801 to a desired position within a vessel V (e.g., proximal to a clot C). Once a distal end of the catheter 807 is in the desired position, the stent and expansion member 801 can be advanced out of the first lumen of the catheter 807 at through or at least partially though the clot C such that a distal portion of the expansion member 801 is distal to the clot C. The stent and the expansion member 801 can be transitioned to an expanded configuration that places the stent in contact with the clot C and the distal portion of the expansion member 801 in contact with an inner wall of the vessel V distal to the clot C. As such, a portion of the vessel V proximal to the distal portion of the expansion member 801 can be aspirated (e.g., via a second lumen of the catheter 807) allowing for the removal of the clot C and a restoration of blood flow through the vessel V.

In the embodiment shown in FIG. 25, the catheter 807 can be a multi-lumen catheter that defines a first lumen for delivering/retrieving the stent (not shown) and the expansion member 801, a second lumen for aspirating the vessel V, and a third lumen that can be configured to receive a guidewire catheter 809. In other embodiments, the catheter 807 can include and/or can form a guidewire catheter or extended portion, which in turn, defines the third lumen of the catheter 807. The guidewire catheter 809 is configured to be advanced from the catheter 807 through the clot C in the vessel V. Moreover, the expansion member 801 is coupled to and/or otherwise formed by a distal portion of the guidewire catheter 809 such that extending the guidewire catheter 809 through the clot C, places the expansion member 801 (or at least a distal portion thereof) in a distal position relative to the clot C. In this embodiment, the expansion member 801 is an atraumatic cap as described above with reference to the expansion member 401. Accordingly, the expansion member 801 can be transitioned from a collapsed state (e.g., as the guidewire catheter 809 and/or expansion member 801 is advanced through the clot C) to an expanded state such that the cap contacts an inner wall of the vessel V.

As shown, a portion of the guidewire catheter 809 extends through the distal cap of the expansion member 801. In some embodiments, the guidewire catheter 809 can form and/or can be integrated with the expansion member 801 such that a seal is present between the guidewire catheter 809 and the distal cap. In other embodiments, the expansion member 801 can define a central opening or the like that is allows the expansion member 801 to be advanced over the guidewire catheter 809. In such embodiments, the opening can be sized such that a seal is formed between the expansion member 801 and an outer surface of the guidewire catheter 809. In some embodiments, the expansion member 801 can include and/or can be coupled to a seal member, o-ring, grommet, etc. configured to form a seal with the outer surface of the guidewire catheter 809. Thus, the expansion member 801 in the expanded state can block, occlude, fill, and/or otherwise seal a portion of the vessel V distal to the clot C, thereby limiting and/or substantially preventing clot fragments from flowing through the vessel V, outside of the guidewire catheter 809, and distal to the expansion member 801.

As shown in FIG. 25, the catheter 807 defines an opening 808 that is in fluid communication with a lumen of the guidewire catheter 809 and/or the third lumen of the catheter 807. As such, advancing the guidewire catheter 809 through the clot C the results in at least partial recanalization of the vessel V prior to advancing and/or deploying the stent. In some instances, the ability to at least partially restore blood flow through the vessel V as quickly as possible can be desirable as delay can result in further tissue damage (e.g., in the case of cerebral arteries when treating patients who suffered ischemic stroke). In addition, the system 800 optionally includes a proximal balloon 810 that is disposed around the multi-lumen catheter 807 and distal to the opening 808. Thus, the opening 808 can provide clean blood that can reduce microemboli in the vessel V distal to the clot C being treated.

FIG. 26 is a schematic side view illustration of a stent 904 according to an embodiment. The stent 904 can be any suitable shape, size, and/or configuration. In some embodiments, the stent 904 and/or aspects or portions thereof can be similar to any of the stents 104, 204, and/or 304 (and/or aspects or portions thereof). For example, the stent 904 can be a self-expanding stent formed from a shape-memory alloy and having an expanded state in which a diameter of a proximal end portion 913 of the stent 904 is smaller than a diameter of a distal end portion 914 of the stent 904. Furthermore, the stent 904 includes, forms, and/or defines a set of cells 915 that selectively can be sized and/or configured to result in a desired characteristic of at least a portion of the stent 904 in the expanded state, as described in detail above with reference to any of the stents 104, 204, and/or 304. Accordingly, such aspects and/or portions of the stent 904 are not described in further detail herein.

The stent 904 shown in FIG. 26 can differ, however, in the shape, size, and/or configuration of the distal end portion 914 of the stent 904. Specifically, the distal end portion 914 of the stent 904 includes and/or forms a flange, flared portion, conical portion, and/or an otherwise enlarged portion. As shown, for example, the stent 904 can have a taper along a length of the stent 904 between the proximal end portion 913 and the distal end portion 914. The distal end portion 914, in turn, forms a flared region 931 (e.g., a cone or the like) that increases in diameter in the direction of the distal end. Moreover, the stent 904 includes a compliant member 932 at or near the distal end of the flared region 931. In some embodiments, the stent 904 includes and/or forms a metal frame, with an edge that may result in trauma to vessel walls and/or other devices. Accordingly, the compliant member 932 can be an atraumatic cap, ring, etc. that can be coupled to and/or disposed about a distal edge of the flared region 931 of the stent 904.

In some implementations, the flared region 931 of the stent 904 can be configured to exert an increased amount of pressure (relative to the rest of the stent 904) on a surface of a portion of the clot. As such, the flared region 931 can engage the clot and can exert a pressure on the clot when the stent 904 is expanded, which in some instances, can urge the clot to move in a proximal direction (e.g., toward a catheter providing aspiration). Moreover, in some embodiments, the flared region 931 can be shaped in a manner that further facilitates proximal movement of the clot (e.g., curved or trumpet-like, and/or any other suitable shape). In some implementations, the stent 904 can be positioned relative to the clot such that at least the compliant member 932 is distal to the clot. In such implementations, the stent 904 can be configured such that the compliant member 932 engages and/or contacts an inner wall of the vessel. As such, the flared region 931 and the compliant member 932 coupled thereto can block a lumen of the vessel distal to the clot in a manner similar to that of the expansion members 101, 201, 401, 501, and/or 801.

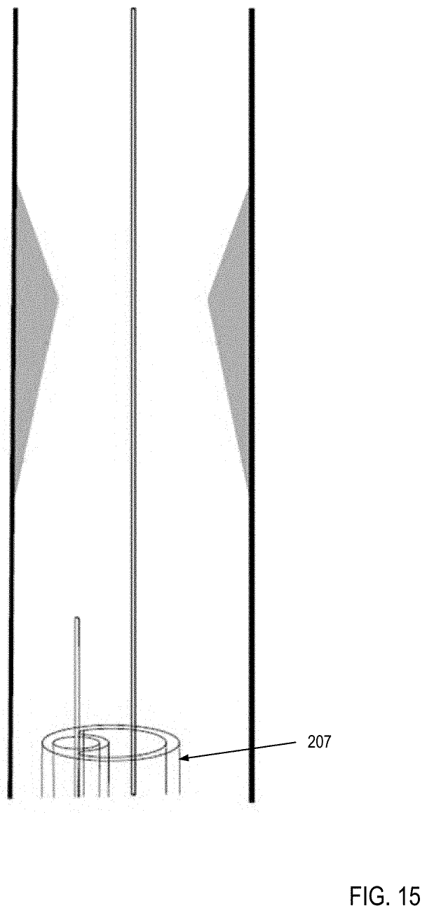

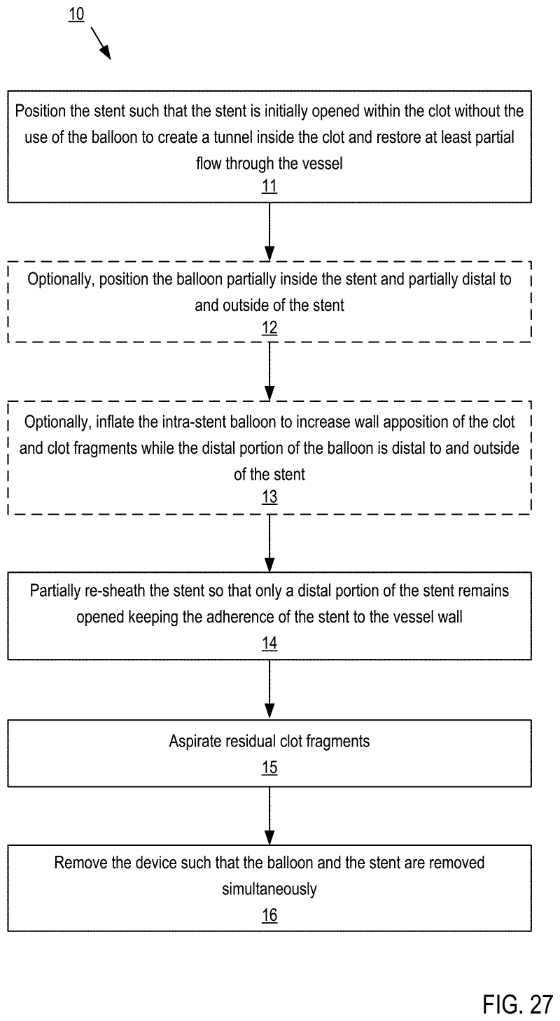

FIG. 27 is a flowchart illustrating an example of a method 10 of using a temporary endovascular clot bypass or thrombectomy system and/or device (such as the device 200 shown in FIGS. 3-15) can be performed in several phases. In some instances, one or more of the several phases can be optional and/or can be performed concurrently with one or more other phases. In some instances, a doctor can design a procedure having any of the phases described below based at least in part on certain characteristics of a given case (e.g., clot type, size, hardness, resistance, etc.).

In some implementations, a first phase can include a positioning of the stent 204 such that the stent 204 is initially opened within the clot (or within the vessel at a clot location) without the use of the balloon 201 to create a tunnel inside the clot and restore at least partial flow through the vessel, at 11. The second phase optionally can include positioning the balloon 201 partially inside the stent 204 and partially distal to and outside of the stent 204, at 12. For example, the operator can position the balloon 201 if the treated artery shows a tendency to occlude again, if the clot is relatively hard, if the operator wants to conclude the intervention and remove the stent 204, and/or the like. The third phase can include intra-stent balloon inflation (e.g., from a deflated state to an inflated state) to increase the wall apposition of the clot and fragments of the clot while the distal portion of the balloon 201 is distal to and outside of the stent 204, at 13. In this implementation, the distal portion of the balloon 201 is beyond the clot to block the migration of emboli during clot PTA. In some instances, the positioning (the second phase, at 12) and/or the inflation of the balloon 204 (the third phase, at 13) need not be performed (e.g., when the clot is relatively soft).

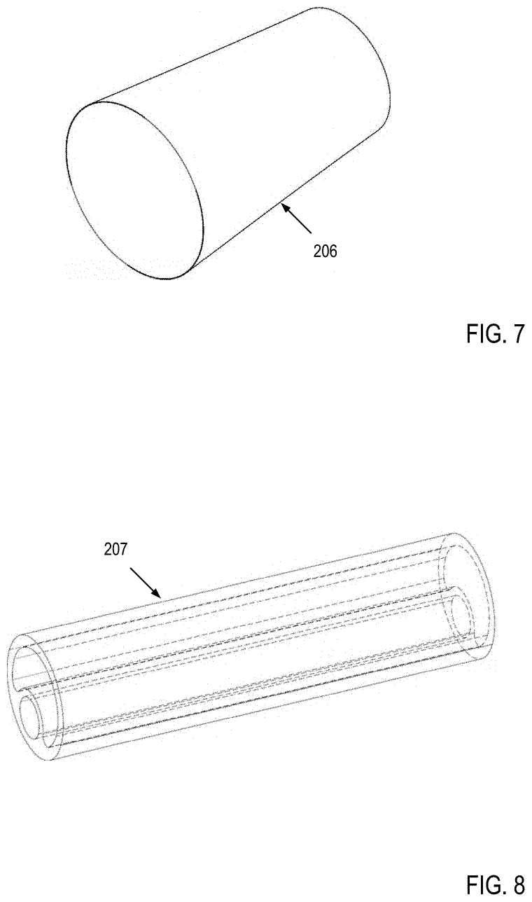

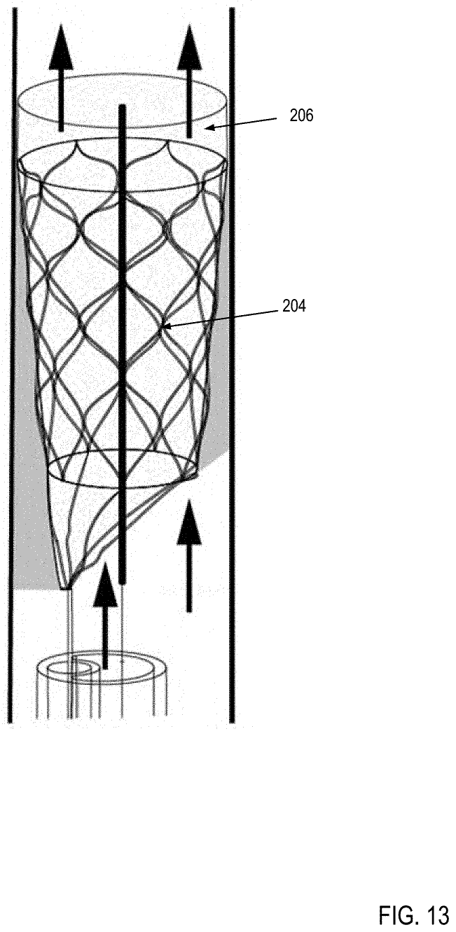

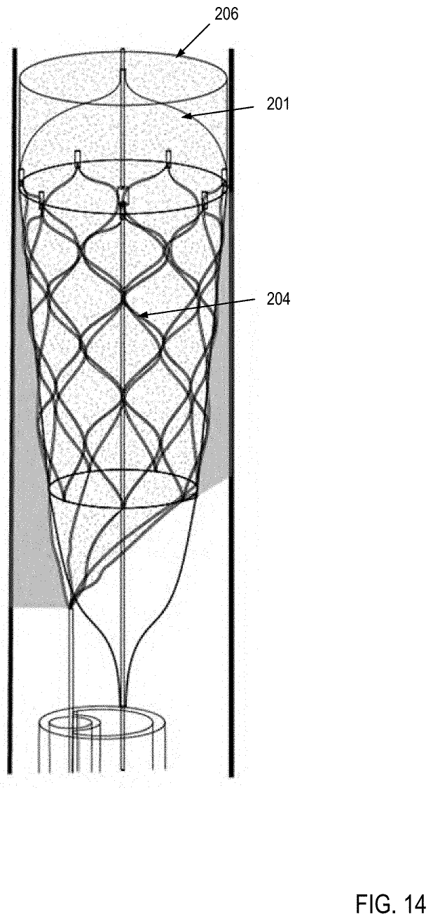

The fourth phase can include a clot trapping process in which the stent 204 is partially re-sheathed so that only the distal portion of the stent (e.g., while the distal end of the balloon 201 is still at least partially inflated) remains opened keeping the adherence of the stent to the vessel wall, at 14. In this way, the membrane or filter 206 of or coupled to the stent 204 and the distal end of the inflated balloon 201 block the migration of clot fragments. In some instances, the fourth phase can include any number of repeated trapping processes. The fifth phase can include an aspiration process in which residual clot fragments (e.g., adherent to the vessel wall) are aspirated, at 15. In some implementations, for example, the clot and/or clot fragments are aspirated through the aspiration lumen of the catheter 207, see e.g., FIG. 7. Aspiration may be done with or without balloon inflation. In this phase, partial opening and partial re-sheathing of the stent 204 and/or partial deflation/inflation of the balloon 201 can be repeated several times while a continuous aspiration is maintained. For example, FIG. 13 shows the stent 204 in an at least partially opened configuration and with the balloon 201 deflated and/or removed, while FIG. 14 shows the opened stent 204 with the balloon 201 inflated. As such, the fourth phase and the fifth phase can be performed sequentially and/or concurrently. The sixth phase can include removal of the device 200 in which the balloon 201 and the stent 204 are removed simultaneously, at 16.

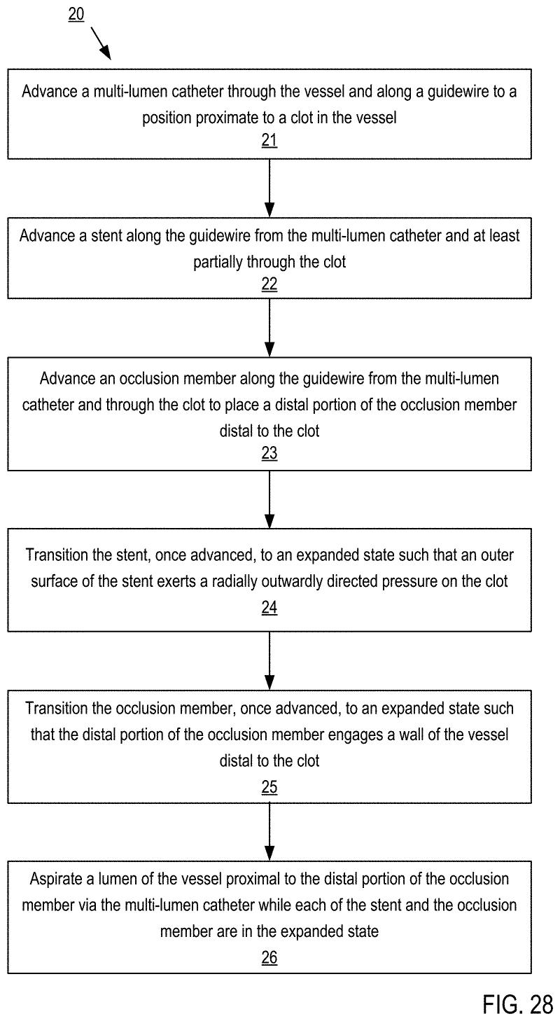

FIG. 28 is a flowchart illustrating an example of a method 20 of using a temporary endovascular clot bypass or thrombectomy system and/or device (“system”) to restore blood flow through a vessel in a body, according to an embodiment. The system can be similar to and/or substantially the same as any of the systems described herein (e.g., the systems 100, 200, 300, and/or 800). For example, in some embodiments, the system can include at least a stent and an expansion member that can be delivered (via a multi-lumen catheter) to a target location in the vessel associated with a clot. In some instances, the vessel can be a cerebral artery in which the clot has led to ischemic stroke. In other instances, the vessel can be any artery (e.g., ex-cerebral arteries) in the body.

The method 20 includes advancing the multi-lumen catheter through the vessel and along a guidewire to a position proximate to the clot in the vessel, at 21. In some implementations, the multi-lumen catheter can be substantially similar to the multi-lumen catheters 107, 207, and/or 807. As such, the multi-lumen catheter can define at least a first lumen and a second lumen.

After positioning the multi-lumen catheter, a stent is advanced along the guidewire from the multi-lumen catheter and at least partially through the clot, as 22. For example, in some implementations, the stent is advanced through and/or from the first lumen of the multi-lumen catheter and into or through the clot. The stent can be and/or can form a wire frame or structure that defines a set of openings or cells and can be substantially similar to the stents 104, 204, and/or 304 described in detail above. An expansion member is advanced along the guidewire from the multi-lumen catheter and through the clot to place a distal portion of the expansion member distal to the clot, at 23. For example, in some implementations, the expansion member is advanced through and/or from the first lumen of the multi-lumen catheter and through the clot. In some instances, the advancing of the stent and the advancing of the expansion member can be performed concurrently in a parallel process. In other instances, the expansion member can be advanced after advancing the stent. Moreover, the advancing of the expansion member is such that the expansion member extends through an interior of the stent, as described above with reference to the systems 100 and/or 200.

After being advanced, the stent is transitioned to an expanded state such that an outer surface of the stent exerts a radially outwardly directed pressure on the clot, at 24. The stent can be transitioned from a collapsed state to the expanded state in response to any suitable actuation. In some implementations, the stent is formed of a shape-memory alloy such that the stent is self-expanding. In such implementations the stent can be transitioned from the collapsed state to the expanded state by removing a force otherwise maintaining the stent in the collapsed state (e.g., a sheath or delivery member, and/or any other suitable member).

The expansion member, after being advanced, is transitioned an expanded state such that a distal portion of the expansion member engages a wall of the vessel distal to the clot, at 25. The expansion member can be substantially similar to any of the expansion members 101, 201, 401, 501, and/or 801 described herein. For example, in some implementations, the expansion member can be and/or can include a balloon, as described above with reference to the expansion member 201. In such implementations, the balloon can be inflated to transition to the expanded state. Moreover, in some implementations, the inflation of the balloon can be operable to and/or can otherwise result in the transitioning of the stent to the expanded state. For example, in some implementations, the balloon can be inflated, which in turn, expands the stent through which the balloon extends, as described above with reference to FIGS. 9-12. In other implementations, the expansion member can be and/or can include an atraumatic distal cap, as described above with reference to the expansion members 401 and/or 801. In such implementations, the expansion member can be transitioned to the expanded state independent of (e.g., after) the stent is transitioned to the expanded state. With the expansion member in the expanded state, the distal portion contacts and/or engages the inner wall of the vessel, thereby occluding the vessel distal to the clot, which can limit and/or substantially prevent clot fragments from flowing through the vessel distal to the expansion member.

A lumen of the vessel proximal to the distal portion of the expansion member is aspirated via the multi-lumen catheter while each of the stent and the expansion member is in the expanded state, at 26. For example, in some implementations, the second lumen of the multi-lumen catheter can be used to provide aspiration of the vessel. As such, the clot or at least a portion of the clot can be drawn into the multi-lumen catheter. In some instances, aspiration can continue after the suctioning of the clot while the stent and the expansion member are transitioned from the expanded state to the collapsed state and retracted, for example, into a first lumen of the multi-lumen catheter. In some instances, providing continued aspiration can limit and/or substantially prevent any clot fragments dislodged during the retraction of the stent and/or expansion member. After retracting the stent and the expansion member, the multi-lumen catheter can be withdrawn from the vessel, through which blood flow has been restored by the bypassing and/or removal of the clot.

In some implementations, the use of a temporary endovascular clot bypass or thrombectomy system and/or device such as, for example, any of the devices 100, 200, 300, and/or 800 described above, can include the following numbered steps.

1. Trapping of the clot between the artery and stent outer surface is started by the balloon inflation pushing the clot backward to eliminate the risk of distal embolization from the non-occluded distal part of the artery.

2. Simultaneous recanalization is achieved after inflating the balloon totally and the whole stent completely expanded to open the lumen and then the balloon is deflated, and distal perfusion is created.

3. Preventing (the risk of distal embolization by the conical shape of the design after balloon inflation at the non-occluded part of the artery the distal end of the balloon) any embolic particle which might migrate distally during fragmentation of the clot by stent expansion, the conical shape of the stent design after balloon inflation pushes the clot backward between the stent and artery wall to be aspirated by the catheter.

4. The single-pass technique is applied with the smart and two diameter mismatch design of the stent of the perfusion created if the stent strength is enough to open clot individually. Otherwise, the balloon will be advanced and inflated (e.g., from a deflated state to an inflated state) to open the occlusion by the hydraulic pressure created by balloon expansion. For this procedure, there is no need to change and catheter or device to go on further steps.

5. To perform an aspiration to retrieve all the clots simultaneously without any distal embolization during stent expansion and balloon inflation, the distal side of the artery is blocked by the distal part of the inflated balloon that prevents distal embolization.

6. Aspiration during removal (a contraction of the stent diameter) of the stent will aspirate all clots trapped between the vessel wall and the stent's external surface.

7. Detachable pusher cable for constant implantation of the stent is used. If there is stenosis under the clot formation, the special design stent which can be fully expanded with the high-pressure balloon, the conical form of the stent returns to a standard stent form and then the connected wire would be detached to leave the stent at the location as an implant.

8. A free distal prolongation of the stent-graft, a curtain-like membrane on the distal end of the stent without metallic frame will be placed as a flow driven filter.

9. The system presents more efficient thrombus removal. After inflating the distal end of the balloon to block the distal embolization, the clot is moved backward, the trapped part is moved by inflation of the balloon and this can be repeated as much as needed to move all the clot back to the aspiration catheter location.

10. Protecting the internal lumen of the stent from clot migration, the flow driven filter can be attached to and/or form at least a portion of an inner surface of the stent. The flow driven filter is a unique design to block the clot between the vessel wall and the stent surface blocking any thrombus migration to the internal surface of the stent covered section of the artery. Block thrombus migration to the internal lumen can allow the balloon to be deflated and re-inflated any number of times.

11. The expansion technique and the deployment of certain components (e.g., the balloon) depends on the resistance, softness, hardness, etc. of the clot. The system gives the physician the ability to create a strategy and/or determine the desired technique after the system is placed into intended location, according to the characterization of the case.

While various embodiments and/or features have been particularly shown and described, it should be understood that they have been presented by way of example only, and not limitation. Various changes in form and/or detail may be made without departing from the disclosure and/or without altering the function and/or advantages thereof unless expressly stated otherwise. Where schematics and/or embodiments described above indicate certain components arranged in certain orientations or positions, the arrangement of components may be modified.

Although various embodiments have been described as having particular features and/or combinations of components, other embodiments are possible having a combination of any features and/or components from any of embodiments described herein, except mutually exclusive combinations. The embodiments described herein can include various combinations and/or sub-combinations of the functions, components, and/or features of the different embodiments described.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

What is claimed:

1. A system, comprising:
a stent forming an annular wall defining a plurality of openings, the stent configured to be advanced into a clot within a vessel of a human body and transitioned to an expanded state such that the annular wall engages at least a portion of the clot; and
an expansion member configured to extend through an interior of the stent such that a distal portion of the expansion member is distal to a distal end of the stent, the expansion member configured to transition to an expanded state after extending through the stent such that a diameter of the distal portion of the expansion member is greater than a diameter of a proximal portion of the expansion member, the distal portion of expansion member in the expanded state being sized to fill a portion of a lumen of the vessel to prevent clot fragments from flowing through the vessel distal to the distal portion of the expansion member.

2. The system of claim 1, further comprising:
a filter coupled to a distal portion of the stent, the filter configured to trap clot fragments when the expansion member is transitioned from the expanded state to a collapsed state.

3. The system of claim 1, further comprising:
at least one catheter collectively having at least a first lumen for advancing the stent and the expansion member to the clot within the vessel and a second lumen for providing continuous aspiration of the lumen of the vessel proximal to the distal portion of expansion member in the expanded state,
the stent and the expansion member configured to be retracted into the first lumen after recanalization of the vessel.

4. The system of claim 1, further comprising:
a multi-lumen catheter having a first lumen for advancing the stent and the expansion member from the multi-lumen catheter to the clot within the vessel, a second lumen for providing aspiration of the lumen of the vessel proximal to the distal portion of the expansion member in the expanded state, the stent and the expansion member configured to be retracted into the first lumen after recanalization of the vessel, and
the multi-lumen catheter having a third lumen for advancing a probe from the multi-lumen catheter into the vessel, the probe having at least one sensor configured to sense a flow of blood through the vessel after recanalization.

5. The system of claim 1, wherein the stent is coupled to a least one sensor configured to sense a flow of blood through the vessel.

6. The system of claim 1, wherein the expansion member is a balloon configured to transition between an inflated state and an uninflated state, a diameter of a distal portion of the balloon in the inflated state being greater than a diameter of a proximal portion of the balloon.

7. The system of claim 1, wherein the distal portion of the expansion member forms an atraumatic cap configured to transition between an expanded state and a collapsed state, the atraumatic cap having a substantially parabolic cross-sectional shape in the expanded state.

8. The system of claim 1, wherein the distal portion of the expansion member forms an atraumatic cap configured to transition between an expanded state and a collapsed state, the atraumatic cap having a substantially parabolic cross-sectional shape in the expanded state,
the expansion member being such that a proximal force exerted along the expansion member in the expanded state is operable to move the atraumatic cap along a wall of the vessel toward the multi-lumen catheter.

9. A system, comprising:
at least one catheter collectively having at least a first lumen and a second lumen;
a stent forming a plurality of cells, the stent transitionable between a collapsed state allowing the stent to be advanced out of the first lumen and at least partially through a clot and an expanded state in which an outer surface of the stent engages at least a portion of the clot; and
an expansion member configured to extend out of the first lumen and through an interior of the stent such that a distal portion of the expansion member is distal to a distal end of the stent, the expansion member configured to transition to an expanded state to engage a wall of the vessel distal to the clot thereby preventing clot fragments from flowing through the vessel distal to the distal portion of the expansion member, and
the second lumen allowing continuous aspiration of a lumen of vessel proximal to the distal portion of the expansion member when each of the stent and the expansion member is in the expanded state.

10. The system of claim 9, wherein the expansion member is a balloon configured to transition between an inflated state and an uninflated state, a diameter of a distal portion of the balloon in the inflated state being greater than a diameter of a proximal portion of the balloon.

11. The system of claim 9, wherein the expansion member is an atraumatic cap configured to transition between an expanded state and a collapsed state, the atraumatic cap having a substantially parabolic cross-sectional shape in the expanded state.

12. The system of claim 9, wherein the stent having at least a distal cell segment including a first subset of cells from the plurality of cells and a proximal cell segment including a second subset of cells from the plurality of cells, the cells in the first subset cells differing from the cells in the second subset of cells in at least one of a cell size or a cell shape such that when the outer surface of the stent engages the clot, the outer surface of the stent corresponding to the distal cell segment exerts a first pressure on a first portion of the clot and the outer surface of the stent corresponding to the proximal cell segment exerts a second pressure on a second portion of the clot less than the first pressure.

13. The system of claim 9, wherein the stent includes:
a first portion having a first set of cells from the plurality of cells, the first set of cells having a first size such that the outer surface along the first portion of the stent exerts a first pressure on the clot when the stent is in the expanded state;
a second portion distal to the first portion and having a second set of cells from the plurality of cells, the second set of cells having a second size greater than the first size such that the outer surface along the second portion of the stent exerts a second pressure on the clot when the stent is in the expanded state, the second pressure being greater than the first pressure; and
a third portion disposed between the first portion and the second portion, the third portion operable to increase a flexibility of at least a portion of the stent between the first portion and the second portion.

14. The system of claim 9, wherein the stent includes:
a first portion having a first set of cells from the plurality of cells, the first set of cells having a first size such that the outer surface along the first portion of the stent exerts a first pressure on the clot when the stent is in the expanded state;
a second portion distal to the first portion and having a second set of cells from the plurality of cells, the second set of cells having a second size greater than the first size such that the outer surface along the second portion of the stent exerts a second pressure on the clot when the stent is in the expanded state, the second pressure being greater than the first pressure;
a third portion disposed between the first portion and the second portion, the third portion operable to increase a flexibility of at least a portion of the stent between the first portion and the second portion;
a fourth portion distal to the second portion and having a third set of cells from the plurality of cells, the third set of cells having a third size greater than the second size such that the outer surface along the fourth portion of the stent exerts a third pressure on the clot when the stent is in the expanded state, the third pressure being greater than the second pressure; and
a fifth portion disposed between the second portion and the fourth portion, the fifth portion operable to increase a flexibility of at least a portion of the stent between the second portion and the fourth portion.

15. A method for restoring blood flow through a vessel in a body, the method comprising:
advancing at least one catheter through the vessel to a position proximate to a clot in the vessel, the at least one catheter collectively having at least a first lumen and a second lumen;
advancing a stent along a guidewire, out of the first lumen, and at least partially through the clot;
advancing an expansion member along the guidewire, out of the first lumen, and through the clot to place a distal portion of the expansion member distal to the clot;
transitioning the stent, after being advanced, to an expanded state such that an outer surface of the stent exerts a radially outwardly directed pressure on the clot;
transitioning the expansion member, after being advanced, to an expanded state such that the distal portion of the expansion member engages a wall of the vessel distal to the clot;
aspirating a lumen of the vessel proximal to the distal portion of the expansion member via the second lumen while each of the stent and the expansion member are in the expanded state.

16. The method of claim 15, wherein the expansion member is a balloon, the transitioning the expansion member to the expanded state including inflating the balloon to an inflated state, a distal portion of the balloon in the inflated state having a diameter greater than a diameter of a proximal portion of the balloon in the inflated state, the distal portion of the balloon in the inflated state engaging the wall of the vessel distal to the clot.

17. The method of claim 15, wherein the catheter is a multi-lumen catheter defining the first lumen and the second lumen:
the advancing the stent includes advancing the stent from the first lumen of the multi-lumen catheter;
the advancing the expansion member includes advancing the expansion member from the first lumen of the multi-lumen catheter; and
the aspirating of the lumen of the vessel includes aspirating the lumen of the vessel via the second lumen of the multi-lumen catheter.

18. The method of claim 15, wherein:
the advancing the stent and the advancing the expansion member being such that the stent and the expansion member are advanced substantially concurrently, the expansion member being partially disposed in the stent such that the distal portion of the expansion member is distal to the stent after the advancing; and
the transitioning the stent and the transitioning the expansion member to the expanded states being such that the stent and the expansion member are transitioned substantially concurrently.

19. The method of claim 15, wherein the at least one catheter collectively has at least the first lumen, the second lumen, and a third lumen, the method further comprising:
advancing a probe out of the third lumen and into the vessel;
sensing, via at least one sensor of the probe, a flow of blood through the vessel prior to the advancing the stent out of the first lumen and through the clot; and
sensing, via the at least one sensor of the probe, a flow of blood through the vessel after the aspirating the lumen of the vessel via the second lumen.

20. The method of claim 15, further comprising:
advancing a probe from the at least one catheter and into the vessel after the aspirating the lumen of the vessel;
energizing a heat source of the probe after advancing the probe into the vessel;
sensing a heat distribution relative to the heat source to determine at least one characteristic associated with a flow of blood through the vessel.
